(19) United States
(12) Patent Application Publication
(10) Pub. No.: US 2008/0241839 A1
(43) Pub. Date: Oct. 2, 2008

(54) Method for correlating differential brain images and genotypes; genes that correlate with differential brain images

(75) Inventors: Steven Potkin, Irvine, CA (US); James H. Fallon, Irvine, CA (US); Fabio Macciardi, Milano (IT); Jessica Turner, Irvine, CA (US); David Keator, Ladera Ranch, CA (US)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA (US)

(21) Appl. No.: 11/974,516

(22) Filed: Oct. 12, 2007

Related U.S. Application Data
(60) Provisional application No. 60/851,379, filed on Oct. 12, 2006.
(60) Provisional application No. 60/855,006, filed on Oct. 27, 2006.

(51) Int. Cl. C12Q 1/68

(52) U.S. Cl. 435/6

(57) ABSTRACT

Methods of assigning quantitative phenotype measurement summary statistics to differential brain image information associated with neuropsychiatric disorders are provided. Summary statistics are correlated to genotype information to identify loci that correlate with differential brain image phenotypes. Methods of identifying modulators of genes at the loci are provided, as well as modulators identified by the methods. Systems for correlating polymorphisms and differential brain image phenotypes, for identifying modulators and for making correlations between differential brain activation phenotypes and genotypes are also provided_{af}.

### CROSS-REFERENCE TO RELATED APPLICATIONS

This application claims priority to and benefit of U.S. provisional patent applications U.S. Ser. No. 60/851,379, filed Oct. 12, 2006 and U.S. Ser. No. 60/855,006, filed Oct. 27, 2007, each of which is incorporated by reference in its entirety.

### FIELD OF THE INVENTION

This invention is in the field of brain imaging and genetic correlations with neuropsychiatric disorders.

### BACKGROUND OF THE INVENTION

The underlying genetic architecture of a quantitative trait is defined by parameters within or among populations. These parameters include the number of quantitative trait loci (QTL) that affect the trait, the frequencies of alternative polymorphisms at the relevant QTL, the patterns of linkage disequilibrium among the QTL and the magnitude of any effects of the QTL (e.g., additive effects, dominance effects and epistatic effects) on the trait. Understanding which QTL influence a trait, and to what degree, has broad applications in biology, including in molecular medicine (e.g., diagnostics, prognostics, and medical treatment options and outcomes), agriculture (e.g., marker assisted selection (MAS)), and in studies directed towards understanding the biological basis for and evolution of the trait. See, e.g., Lynch and Walsh (1998) Genetics and Analysis of Quantitative Traits Sinauer and associates, Inc. Sunderland, Mass.

At least three fundamentally different approaches are used to study gene-phenotype interactions. In the first, most common in molecular medicine, a “candidate gene” approach is used. In this approach, knowledge about a gene's biological activity (or likely biological activity, e.g., based on homology) is used to form an hypothesis regarding a relationship between a gene and a trait. Any proposed association can be studied using any of a variety of statistical or analytical methods to determine whether the gene influences the phenotype. This approach is severely limited, because of the requirement for a priori knowledge regarding a gene's function, before its correlation with phenotype can be studied.

The second basic approach to identifying gene phenotype interactions is to screen the genomes of individuals, e.g., with a whole genome scan, to identify genetic differences between individuals, in an attempt to identify which genetic differences influence observed phenotypic differences between the individuals. This approach, which is common, e.g., in agriculture, requires large sample sizes and standard genetic backgrounds for the individuals in the population to establish a reasonable statistical correlation between the gene and the phenotype. Such methods are often not feasible for screening populations with diverse genetic backgrounds, and small sample sizes, such as typically occurs when considering human populations.

A third approach, also common in agriculture, is to rely on extremely detailed linkage maps generated by classical genetic methods to identify regions of a chromosome that encode a trait. The region can be cloned via positional cloning and analyzed for candidate genes, which can be tested as noted above. This approach is labor intensive and requires extremely detailed linkage maps, which may not exist for all regions of interest.

All of the above methods also share a further limitation, in that pre-existing detailed knowledge about the distribution of the phenotype of interest is a prerequisite to discovering gene associations. This is particularly problematic for many traits that are highly complex and difficult to quantify. For example, while psychiatric disorders such as schizophrenia and bipolar disorder can be diagnosed with reasonable accuracy, these disorders have many disparate symptoms, etiologies, and presentations. They likely have several distinct biological and environmental causes, or potential causes. Thus, the assignment of separate phenotypes for different types of schizophrenia, or other forms of mental illness, is highly challenging.

The present invention overcomes these and other difficulties, providing a robust method of assigning phenotypes to differences in brain function and of determining correlations between genes and these phenotypes. This, in turn, is extremely valuable in molecular medicine, e.g., for the diagnosis, prognosis and treatment of individuals that suffer from various complex disorders, particularly neuropsychiatric disorders.

### SUMMARY OF THE INVENTION

The invention includes general methods for identifying markers that correlate with neuropsychiatric disorders such as schizophrenia, bipolar disorder, etc., as well as several markers/genes that were identified by these methods.

In a first aspect, methods of characterizing differential brain activation are provided. The methods include measuring a brain image under a first functional condition, measuring a brain image under a second functional condition, and determining a difference between the brain image under the first and second condition. A summary statistic is then assigned to the difference. This summary statistic is used as a description of a differential brain activation phenotype, which can then be correlated with genotype differences. Images can be provided using any of a variety of technologies, including MRI, PET scanning and the like. For example, the brain image can include an fMRI brain scan of the patient, e.g., for a functional MRI test of the normal and abnormal patients (e.g., a working memory test).

Accordingly, in a related aspect, methods of correlating a brain image phenotype to a genotype are provided. The methods include detecting variance in a brain image phenotype (e.g., determined using the summary statistic as noted above) in at least one population, accessing genotype information for the population, and correlating the variance to the genotype information, thereby correlating the brain image phenotype and the genotype.

The population typically comprises a group of cogitatively and psychiatrically healthy individuals and a group of patients that suffer from a neuropsychiatric disorder, with the variance being a difference in brain image phenotype between the normal individuals and the patients. For example, the group of patients can include patients that are schizophrenic or that suffer from a bipolar or other neuropsychiatric disorder. The variance in the brain image phenotype comprises a variance in differential brain activation between members of the population, e.g., measured using a summary statistic as noted above.

For example, detecting variance in a brain image phenotype optionally comprises assigning a summary statistic for an image for at least one region of the brain for at least one member of the population. Assigning the summary statistic optionally includes: measuring a first brain image of a brain region under a first functional condition; measuring a second brain image of the brain region under a second functional condition; determining a difference between the first and second brain image; and, assigning the summary statistic to reflect the difference. For example, the first brain image and the second brain image are optionally extracted from a corresponding first and second brain scan using a Talairach or MNI atlas, with the summary statistic reflecting a difference between an observed brain image for a brain engaged in a high memory task and an observed brain image for a brain engaged in a low memory task for the at least one region. Optionally, the at least one region of the brain is a well characterized structural or functional region, e.g., the left hemisphere Broadman Area 46, DLPFC BA-9, DPFC, BA 6 the Premotor Cortex, the Dorsal Premotor Cortex, BA 7 (Superior Parietal Lobule), BA 8 Frontal Eye Field/Premotor Cortex, posterior dorsal prefrontal cortex, BA24 (Left Anterior Cingulate), the Left Whole Thalamus, Caudate, Amygdala, and/or the Right Cerebellum.

The genotype information typically comprises a dataset derived from hybridization of a sample to an array of polymorphisms. For example, the genotype information can include SNP data sets for at least about 100,000 representative SNPs for a plurality of members of the population. The variance can be correlated by any statistical method, but in a preferred aspect is correlated using a general linear model (GLM). For example, one preferred GLM assumes that imaging phenotype=overall mean+genotype effect+diagnosis effect+genotype-diagnosis interaction effect. The variance is optionally correlated by performing linear regression to compare image phenotype information across the population to SNP genotype information across the population, wherein the comparison comprises testing for an equality of means across the genotype information, assuming a codominant genetic model that tests for additive effects, dominant effects and effects equal to zero.

Any of a variety of confirmatory analysis can be performed to improve the confidence of any correlation. For example, The methods can include replicating the correlation in an independent sample or population. An additional approach to improving confidence includes correlating the variance to genetically linked polymorphisms using a haplotype correction criterion (linked polymorphisms should display correlation with a trait of a linked QTL). Further, the variance can optionally be correlated to a plurality of genetically linked polymorphisms using a within-study confirmation analysis. Studies that determine whether there is a correlation between genes and phenotypes can also be further verified by determining whether differential activation occurs in functionally/structurally related brain structures. For example, the variance can be a first variance in differential activation in a first region of the brain, and the method include detecting an additional variance in differential activation in an anatomically or functionally connected region of the brain, where the first variance and the additional variance correlate similarly to the genotype information.

In a related aspect, systems for correlating brain image phenotype and genotype are provided. For example, systems can include a brain image scan device, a database of genotype information, and a correlation module that correlates the genotype information to a brain scan produced by the brain scan device.

Features noted above for the methods are applicable to the systems as well (and vice-versa). For example, The correlation module optionally comprises a general linear model (e.g., implemented by system software). The correlation module typically has a database that includes a lookup table that comprises correlation relationships for differential brain scan measurements and the genotype information. This database can be a heuristic database that refines correlations between genotype information and differential brain scan measurements. For example, the heuristic database can include a general linear model (GLM), a neural network (NN), a statistical model (SM), a hidden Markov model (HMM), a principal component analysis (PCA) feature, a classification and regression trees (CART) feature, multivariate adaptive regression splines (MARS), a genetic algorithm (GA), a multiple linear regression (MLR) feature, variable importance for projection (VIP), inverse least squares (ILS), a partial least square (PLS) feature, or the like.

In one preferred aspect of the methods and systems, the method or systems include evaluating the genotype and phenotype information with a general linear model (e.g., implemented with system software) that assumes that phenotype=overall mean+genotype effect+diagnosis effect+genotype-diagnosis interaction effect. In this model, the phenotype can be a resonance imaging phenotype, e.g., a differential brain scan phenotype.

The methods noted above have been used to identify a variety of correlations between genotypes and differential brain image phenotypes, thereby identifying relationships between corresponding neuropsychiatric disorders and the genotypes. Accordingly, an additional aspect of the invention provides methods of identifying a neuropsychiatric disorder predisposition phenotype in a patient. The methods include detecting, in the patient or in a biological sample derived from the patient, a polymorphism in a locus, gene or gene product of Appendix 1, or a polymorphism in a locus closely linked to the Appendix 1 gene or locus. The polymorphism is associated with the neuropsychiatric disorder predisposition phenotype. The method typically also includes correlating the polymorphism to the phenotype.

Preferred examples of genes/loci from Appendix 1 include: LOC148823 [C1orf150], PPP1CB, SPDY1, KIAA1604, MGC42174, NPY5R, SFXN1, ARHGAP18, ZNF297B, MKI67, FLJ22531, PC, and SPINL (SPIN1). ARHGAP18 is a particularly preferred gene that has been shown to correlate with differential brain images associated with schizophrenia.

The phenotype can include or be representative of bipolar disorder, schizophrenia or a related neuropsychiatric disorder, or the like. In one example, the phenotype comprises abnormal prefrontal brain activation, e.g., associated with schizophrenia.

Detection of the polymorphism can include hybridization of a probe to a nucleic acid comprising the polymorphism, locus, or a complementary nucleic acid thereof. In typical embodiments, the detection can include amplifying the polymorphism or a sequence associated therewith and detecting the resulting amplicon. Other detection formats include detecting differences in expression levels (e.g., via northern or western analysis), or direct detection with high signal probes such as branched DNA (bDNA) probes.

The polymorphism can be any detectable polymorphism, including microsatellite DNA, single nucleotide polymorphisms ((SNPs), e.g., a SNP selected from the group consisting of those listed in Appendix 1), or the like. In one specific example, the polymorphism comprises an RS9372944 or RS9385523 SNP. Correlating the polymorphism typically comprises referencing a look up table that comprises established correlations between alleles of the polymorphism and the phenotype.

The closely linked locus is typically about 5 cM or less from the gene, and can be 1 cM, 0.1 cM, or less from the gene. Loci that are more closely linked to a QTL are better markers for the QTL.

The invention further provides systems for correlating the polymorphisms noted above, e.g., similar to the systems previously noted, further including look up tables with established correlations between the loci of appendix 1 and a relevant phenotype. For example, the invention includes systems for identifying a neuropsychiatric disorder predisposition phenotype for a patient, the system comprising: a) a set of marker probes or primers configured to detect at least one allele of one or more gene or linked locus associated with the predisposition phenotype, wherein the gene encodes a gene of appendix 1; b) a detector that is configured to detect one or more signal outputs from the set of marker probes or primers, or an amplicon produced from the set of marker probes or primers, thereby identifying the presence or absence of the allele; and, c) system instructions that correlate the presence or absence of the allele with the predicted phenotype. The set of marker probes typically comprises or hybridizes to a nucleotide sequence provided in Appendix 1. The instructions typically include at least one look-up table that includes a correlation between the presence or absence of the allele and the predisposition phenotype.

Methods of identifying a modulator of a neuropsychiatric disorder phenotype are also a feature of the invention. The method includes contacting a potential modulator to a gene or gene product of Appendix 1; and, detecting an effect of the potential modulator on the gene or gene product, thereby identifying whether the potential modulator modulates the phenotype. All features of the disorder and phenotype noted above are applicable here as well.

The effect to be detected can be any that is logically related to an activity of the gene or gene product, including (a.) increased or decreased expression of the gene in the presence of the modulator; (b.) a change in localization of the gene product in the presence of the modulator; (c.) a change in an activity of a RHO-GTPase encoded by an ARHGAP18 gene; and, (d.) a change in RAS or EGFR-mediated cell proliferation, migration or differentiation (this activity is related to ARHGAP18 activity, as noted in more detail herein). The modulator can be, e.g., a transcription/translation modulator (e.g., an siRNA), a methylation modulator, a histone modulator, a cis site modulator, a secondary messenger, an environmental impact modulator, a stress modulator, nicotine, etc.

In a related aspect, the invention also provides a kit for treatment of a neuropsychiatric disorder. The kit includes a modulator identified by the methods noted above, and instructions for administering the modulator to a patient to treat the disorder.

### BRIEF DESCRIPTION OF THE DRAWINGS

FIG. 1 is a histogram and brain activity image. Peaks of the histogram represent p values (plotted as −log p) for all SNPs represented on the Illumina Human-1 Genotyping Bead Chip over an approximately 10 million basepair region of chromosome 6, with flanking basepair numbers indicated. Each tube represents a different region of brain activation. he specific RS number for SNPs coincident with the main peaks are listed in their approximate locations. The MRI template demonstrates the implied circuitry for brain areas represented in the FIGURE.

### DETAILED DESCRIPTION

Previous candidate gene approaches limit the target gene under consideration to those that have a known biological relationship to the disorder or condition of interest. Contrawise, genome-wide scans (GWS) are plagued by false positive and false negative results, and the requirement for very large, and even unobtainable sample sizes.

The invention includes methods that utilize brain imaging to guide discovery of genes relevant to brain image differences. In the methods herein, we determine brain imaging differences between patient populations and healthy controls, and then determine which genes or genetic variation influence or cause these differences. This method can be used to make more accurate diagnoses and to discover new treatments for brain illnesses.

We initially contrast brain imaging patterns between the patient population and normal healthy controls, to generate summary measures on differential patterns. These patterns can be structural or functional and can include MRI, PET, EEG and MEG measures. A GLM parallel analyses of all genetic variation is calculated with the brain measures as the dependent variable. Genetic variation can include SNPs, haplotypes, blocks, VNTR, microsatellite, sequence data, or the like. The resultant IGPs (imaging genophenotypes) are considered in a hierarchical procedure. Candidate genes determined a priori are first considered with a rigorous correction for the number of tests. Then the remaining SNPs (non-candidate) are considered using appropriate corrections for a larger number of GLM tests. This procedure identifies top candidate genes and IGPs for further analysis.

Any method of correction based on statistical methods brings with it an expected false negative rate. Additional genetic information is expected to protect against false negatives, as well as removing false positives. Therefore, the IGPs that pass the rigorous correction above should be interrogated using a denser SNP and other methods on measuring genetic variation by chip or other methods.

The identified genes from the above analyses are interrogated with a denser polymorphism array to obtain additional information on genotyping in what is a within-study confirmation. This censored analysis is repeated with the additional genetic data. The surviving results are confirmed in an independent sample, which is essentially a between-study confirmation.

Appendix 1 provides correlations between a variety of genes and imaging phenotypes, including associations between differential brain activation in schizophrenic patients and polymorphisms in ARHGAP 18, LOC148823 [C1orf150], PPP1CB, SPDY1, KIAA1604, MGC42174, NPY5R, SFXN1, ARHGAP18, ZNF297B, MKI67, FLJ22531, PC, and SPINL (SPIN1). Further details regarding these genes is available in the literature, e.g., following the links in Appendix 1.

### DEFINITIONS

It is to be understood that this invention is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule. Letter designations for genes or proteins can refer to the gene form and/or the protein form, depending on context. One of skill is fully able to relate the nucleic acid and amino acid forms of the relevant biological molecules by reference to the sequences herein, known sequences and the genetic code.

Unless otherwise indicated, nucleic acids are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “neuropsychiatric disorder” is a disorder comprising neurological and/or psychiatric features. Examples include diseases that affect the brain or the mind, including schizophrenia and other psychotic disorders, bipolar disorder, mood disorders such as major or clinical depression, anxiety disorders such as generalized anxiety disorder, somatoform disorders (Briquet's disorder), factitious disorders such as Munchausen syndrome, dissociative disorders such as dissociative identity disorder, sexual disorders such as dyspareunia and gender identity disorder, eating disorders such as anorexia nervosa, sleep disorders such as insomnia and narcolepsy, impulse control disorders such as kleptomania, adjustment disorders, personality disorders such as narcissistic personality disorder, tardive dyskinesia, tourettes, autism, and many others. The term is used broadly to encompass “neurodiversity” as well as “illness”. “Neurodiversity,” is a concept arguing that atypical neurological wiring is a “normal” human difference rather than an illness per se. This can include, for example, autism, dyslexia, dyspraxia and hyperactivity.

A “brain image” is a representation of brain structure or activity. Examples include brain scanning technologies such as MRI, fMRI, and PET scanning, as well as EEG measurements and other available methods of measuring and recording brain structure or activity.

A “brain image phenotype” is a phenotype that is detected by scanning or otherwise imaging the brain. Typically, this brain image phenotype is determined from scanning or imaging a brain of a living individual. A variety of imaging technologies are available for scanning a living brain, including magnetic resonance imaging (MRI), such as fMRI, in which brain function is monitored, e.g., during specified cognitive activities (e.g., various memory or other cognition tasks). PET, EEG and MEG can also be used for imaging. A “differential brain image phenotype” is a detectable variance between individuals for differential brain images (scans) of the individuals. For example, a first individual's brain can be scanned under a first set of conditions (e.g., during a first cognitive task) and again under a second set of conditions (e.g., during a second cognitive task). The brain images for the first and second set of conditions are different, and the difference between the images can be quantified. This quantified difference can be similarly determined for other individuals, using the same first and second set of conditions. The quantified differences between the individuals provides the phenotypic variance for the overall population of individuals.

A “patient” is typically a human patient to be evaluated or treated, e.g., by a clinician. However, the term also optionally encompasses veterinary (non-human) patients.

A “phenotype” is a trait or collection of traits that is/are observable in an individual or population. The trait can be quantitative (a quantitative trait, or QTL) or qualitative.

A “polymorphism” is a locus that is variable; that is, within a population, the nucleotide sequence at a polymorphism has more than one version or allele. The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. One example of a polymorphism is a “single nucleotide polymorphism” (SNP), which is a polymorphism at a single nucleotide position in a genome (the nucleotide at the specified position varies between individuals or populations). Other typical examples include haplotypes, blocks, VNTR, microsatellite, and sequence data.

An allele “positively” correlates with a trait when it is linked to it and when presence of the allele is an indictor that the trait or trait form will occur in an individual comprising the allele. An allele negatively correlates with a trait when it is linked to it and when presence of the allele is an indicator that a trait or trait form will not occur in an individual comprising the allele.

A marker polymorphism or allele is “correlated” with a specified phenotype (e.g., a differential brain scan phenotype) when it can be statistically linked (positively or negatively) to the phenotype. This correlation is often inferred as being causal in nature, but it need not be—simple genetic linkage to (association with) a locus for a trait that underlies the phenotype is sufficient.

A “favorable allele” is an allele at a particular locus that positively correlates with a desirable phenotype, e.g., resistance a neuropsychiatric disorder, or that negatively correlates with an undesirable phenotype, e.g., an allele that negatively correlates with predisposition to a neuropsychiatric illness. A favorable allele of a linked marker is a marker allele that segregates with the favorable allele. A favorable allelic form of a chromosome segment is a chromosome segment that includes a nucleotide sequence that positively correlates with the desired phenotype, or that negatively correlates with the unfavorable phenotype at one or more genetic loci physically located on the chromosome segment.

An “unfavorable allele” is an allele at a particular locus that negatively correlates with a desirable phenotype, or that correlates positively with an undesirable phenotype, e.g., a positive correlation to a neuropsychiatric disorder predisposition. An unfavorable allele of a linked marker is a marker allele that segregates with the unfavorable allele. An unfavorable allelic form of a chromosome segment is a chromosome segment that includes a nucleotide sequence that negatively correlates with the desired phenotype, or positively correlates with the undesirable phenotype at one or more genetic loci physically located on the chromosome segment.

“Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line or population by averaging the allele frequencies of a sample of individuals from that line or population. Similarly, one can calculate the allele frequency within a population of lines by averaging the allele frequencies of lines that make up the population.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location in the genome of a species where a specific gene can be found. Similarly, the term “quantitative trait locus” or “QTL” refers to a locus with at least two alleles that differentially affect the expression or alter the variation of a quantitative or continuous phenotypic trait in at least one genetic background.

A “marker,” “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. In one aspect, the present invention provides marker loci correlating with a phenotype of interest, e.g., a differential brain scan phenotype. Each of the identified markers is expected to be in close or overlapping physical and genetic proximity (resulting in physical and/or genetic linkage) to a genetic element, e.g., a QTL, that contributes to the relevant phenotype. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of allele specific hybridization (ASH), detection of single nucleotide extension, detection of amplified variable sequences of the genome, detection of self-sustained sequence replication, detection of simple sequence repeats (SSRs), detection of single nucleotide polymorphisms (SNPs), or detection of amplified fragment length polymorphisms (AFLPs).

A “genetic map” is a description of genetic linkage (or association) relationships among loci on one or more chromosomes (or linkage groups) within a given species, generally depicted in a diagrammatic or tabular form. “Mapping” is the process of defining the linkage relationships of loci through the use of genetic markers, populations segregating for the markers, and standard genetic principles of recombination frequency. A “map location” is an assigned location on a genetic map relative to linked genetic markers where a specified marker can be found within a given species. The term “chromosome segment” designates a contiguous linear span of genomic DNA that resides on a single chromosome. Similarly, a “haplotype” is a set of genetic loci found in the heritable material of an individual or population (the set can be a contiguous or non-contiguous). In the context of the present invention genetic elements such as one or more alleles herein and one or more linked marker alleles can be located within a chromosome segment and are also, accordingly, genetically linked, a specified genetic recombination distance of less than or equal to 20 centimorgan (cM) or less, e.g., 15 cM or less, often 10 cM or less, e.g., about 9, 8, 7, 6, 5, 4, 3, 2, 1, 0.75, 0.5, 0.25, or 0.1 CM or less; That is, two closely linked genetic elements within a single chromosome segment undergo recombination during meiosis with each other at a frequency of less than or equal to about 20%, e.g., about 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25%, or 0.1% or less.

A “genetic recombination frequency” is the frequency of a recombination event between two genetic loci. Recombination frequency can be observed by following the segregation of markers and/or traits during meiosis. In the context of this invention, a marker locus is “associated with” another marker locus or some other locus (for example, locus correlating with a phenotype or disorder herein), when the relevant loci are part of the same linkage group due to association and are in linkage disequilibrium. This occurs when the marker locus and a linked locus are found together in progeny more frequently than if the loci segregate randomly. Similarly, a marker locus can also be associated with a trait, e.g., a marker locus can be “associated with” a given trait when the marker locus is in linkage disequilibrium with the trait. The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random frequency (in the case of co-segregating traits, the loci that underlie the traits are in sufficient proximity to each other). Linked loci co-segregate more than 50% of the time, e.g., from about 51% to about 100% of the time. Advantageously, the two loci are located in close proximity such that recombination between homologous chromosome pairs does not occur between the two loci during meiosis with high frequency, e.g., such that closely linked loci co-segregate at least about 80% of the time, more preferably at least about 85% of the time, still more preferably at least 90% of the time, e.g., 91%, 92%, 93%, 94%, 95%, 96%, 97%, 98%, 99%, 99.5%, 99.75%, or 99.90% or more of the time.

The phrase “closely linked,” in the present application, means that recombination between two linked loci (e.g., a SNP such as one identified in Appendix 1 herein and a second linked allele) occurs with a frequency of equal to or less than about 20%. Put another way, the closely (or “tightly”) linked loci co-segregate at least 80% of the time. Marker loci are especially useful in the present invention when they are closely linked to target loci (e.g., QTL for a disorder or phenotype herein or, alternatively, simply other marker loci). The more closely a marker is linked to a target locus, the better an indicator for the target locus that the marker is. Thus, in one embodiment, tightly linked loci such as a marker locus and a second locus display an inter-locus recombination frequency of about 20% or less, e.g., 15% or less, e.g., 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less, and still more preferably about 1% or less. In highly preferred embodiments, the relevant loci (e.g., a marker locus and a target locus such as a QTL) display a recombination frequency of less than about 1%, e.g., about 0.75% or less, more preferably about 0.5% or less, or yet more preferably about 0.25% or less, or still more preferably about 0.1% or less. Two loci that are localized to the same chromosome, and at such a distance that recombination between the two loci occurs at a frequency of less than about 20%, e.g., 15%, more preferably 10% (e.g., about 9%, 8%, 7%, 6%, 5%, 4%, 3%, 2%, 1%, 0.75%, 0.5%, 0.25%, 0.1% or less) are also said to be “proximal to” each other. When referring to the relationship between two linked genetic elements, such as a genetic element contributing to a trait and a proximal marker, “coupling” phase linkage indicates the state where the “favorable” allele at the trait locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for a phenotype or disorder of interest) is physically associated on the same chromosome strand as an “unfavorable” allele at the proximal marker locus, and the two “favorable” alleles are not inherited together (i.e., the two loci are “out of phase” with each other).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

A specified nucleic acid is “derived from” a given nucleic acid when it is constructed using the given nucleic acid's sequence, or when the specified nucleic acid is constructed using the given nucleic acid.

A “gene” is one or more sequence(s) of nucleotides in a genome that together encode one or more expressed molecule, e.g., an RNA, or polypeptide. The gene can include coding sequences that are transcribed into RNA which may then be translated into a polypeptide sequence, and can include associated structural or regulatory sequences that aid in replication or expression of the gene. Genes of interest in the present invention include genomic sequences that encode, e.g.: expression products of the ARHGAP 18 gene, or any other gene or gene product in Appendix 1, including, e.g., LOC148823 [C1orf150], PPP1CB, SPDY1, KIAA1604, MGC42174, NPY5R, SFXN1, ARHGAP18, ZNF297B, MKI67, FLJ22531, PC, and SPINL (SPIN1).

A “genotype” is the genetic constitution of an individual (or group of individuals) at one or more genetic loci. Genotype is defined by the allele(s) of one or more known loci of the individual, typically, the compilation of alleles inherited from its parents. A “haplotype” is the genotype of an individual at a plurality of genetic loci on a single DNA strand. Typically, the genetic loci described by a haplotype are physically and genetically linked, i.e., on the same chromosome strand. An “imaging genotype” is a genotype that correlates with a brain image phenotype.

A “set” of markers or probes refers to a collection or group of markers or probes, or the data derived therefrom, used for a common purpose, e.g., identifying an individual with a specified phenotype (e.g., differential brain activation, etc.). Frequently, data corresponding to the markers or probes, or derived from their use, is stored in an electronic medium. While each of the members of a set possess utility with respect to the specified purpose, individual markers selected from the set as well as subsets including some, but not all of the markers, are also effective in achieving the specified purpose.

A “look up table” is a table that correlates one form of data to another, or one or more forms of data with a predicted outcome to which the data is relevant. For example, a look up table can include a correlation between allele data and a predicted trait that an individual comprising one or more given alleles is likely to display. These tables can be, and typically are, multidimensional, e.g., taking multiple alleles into account simultaneously, and, optionally, taking other factors into account as well, such as genetic background, e.g., in making a trait prediction.

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

A “translation product” is a product (typically a polypeptide) produced as a result of the translation of a nucleic acid. A “transcription product” is a product (e.g., an RNA, optionally including mRNA, or, e.g., a catalytic or biologically active RNA) produced as a result of transcription of a nucleic acid (e.g., a DNA).

An “array” is an assemblage of elements. The assemblage can be spatially ordered (a “patterned array”) or disordered (a “randomly patterned” array). The array can form or comprise one or more functional elements (e.g., a probe region on a microarray) or it can be non-functional.

### Identifying Brain Image Phenotypes

In a first aspect, the invention optionally includes determining brain images and differential brain image phenotypes for individuals. A novel feature of the invention includes the characterization of differences in brain images for different states of an individual with a summary statistic, that is then correlated to genotypic differences between individuals.

A variety of brain scanning/imaging technologies are currently available, widely in use and adaptable to the present invention. These include magnetic resonance imaging (MRI), functional magnetic resonance imaging (fMRI), electroencephalograph (EEG) imaging, magnetoencephalography (MEG) imaging, Computerized Axial Tomography (CAT) scanning/imaging, and Positron Emission Tomography (PET) scanning/imaging. Use of each of these methods in determining images in the context of the invention is briefly discussed below. Further details on the general topic of imaging can be found in the literature, e.g., in Beaumont and Graham (1983) Introduction to Neuropsychology. New York: The Guilford Press; Changeux (1985) Neuronal Man: The Biology of Mind New York: Oxford University Press; Malcom (1994) Mind Fields: Reflections on the Science of Mind and Brain. Grand Rapids, Mich.: Baker Books; Lister and Weingartner (1991) Perspectives on Cognitive Neuroscience. New York: Oxford University Press; Mattson and Simon (1996) The Pioneers of NMR and Magnetic Resonance in Medicine. Dean Books Company; Lars-Goran and Markowitsch (1999) Cognitive Neuroscience of Memory. Seattle: Hogrefe & Huber; Norman (1981) Perspectives on Cognitive Science. New Jersey: Ablex Publishing Corporation; Rapp (2001) The Handbook of Cognitive Neuropsychology. Ann Arbor, Mich.: Psychology Press; Purves et al. (2001) Neuroscience, Second Edition Sinauer Associates, Inc. Sunderland, Mass.; and, The Molecular Imaging and Contrast Agent Database (published on line, current through the present date: http://www.ncbi.nlm.nih.gov/books/bookres.fcgi/micad/home.html).

Magnetic Resonance Imaging (MRI) uses magnetic fields and radio waves to produce dimensional images of brain structures, without the use of ionizing radiation or radioactive materials (radio active tracer dyes, etc.). In MRI, a large magnet creates a magnetic field around the head of the patient, through which radio waves are sent. The magnetic field is superimposed, and each point in space in the head has a unique radio frequency at which the signal is received and transmitted. Sensors read the frequencies and a computer uses the information to construct an image. In the present invention, differences between patient and control individuals, or between patients at a first state and a second state as compared to control individuals at the two states can be used to assign images differences. As discussed herein, a summary statistic can be assigned to represent a relevant image or image difference, with this statistic providing the relevant metric for comparison between individuals in the genotype correlation analysis. The image differences (or summary statistics) are correlated to genotype as noted herein.

Functional Magnetic Resonance Imaging (fMRI) uses the differential paramagnetic properties of oxygenated and deoxygenated hemoglobin to see images of changing blood flow in the brain. These properties are associated with neural activity (greater flow is indicative of activity). This allows images to be generated that reflect which brain structures are activated (and how) during performance of different tasks (memory tasks, vision tasks, association testing, etc.). fMRI systems provide subjects with different visual images, sounds and touch stimuli, and to make different actions such as pressing a button or moving a joystick in response to stimuli. Consequently fMRI is used to reveal brain structures and processes associated with perception, thought, memory and action. fMRI is the most preferred method of making differential images in the present invention. As discussed herein, one feature of the invention is that a summary statistic can be assigned to represent relevant image differences, with this statistic providing the relevant metric for comparison between individuals in the correlation analysis. The image differences (or summary statistics) are correlated to genotype as noted herein.

Computed Tomography (CT) or Computed Axial Tomography (CAT) scanning takes series of x-rays of the head from several different directions, and then recompiles the information. Typically used for quickly viewing brain injuries (e.g., following stroke), CT scanning uses a set of equations to estimate how much an x-ray beam is absorbed in a selected volume of the brain. Typically the information is presented as cross sections of the brain. In the context of the present invention, CT differences between patient and control individuals, or between patients at a first state and a second state as compared to control individuals at the two states can be used to assign images differences. These image differences are correlated to genotype as noted herein.

Positron Emission Tomography (PET) measures emissions from radiolabeled metabolically active compounds that are injected into the bloodstream of the patient. The methods uses data from the emissions to produce dimensional images of the distribution of the chemicals throughout the brain. The labeled compound, typically called a “radiotracer,” is injected into the bloodstream and makes its way to the brain. Sensors in the PET scanner detect the radioactivity as the compound accumulates in different regions of the brain. A computer uses the data gathered by the sensors to create multicolored two or three-dimensional images that show where the compound acts in the brain. One advantage of PET scanning is that different compounds can show blood flow and oxygen and glucose metabolism in the tissues of the working brain. These measurements reflect the amount of brain activity in the various regions of the brain and can be used in a manner similar to fMRI noted above to determine differences in activation patterns for patients and normal controls. Accordingly, PET scanning is another preferred method of making differential images in the present invention. As with fMRI, a summary statistic can be assigned to represent relevant image differences revealed by PET scanning, with this statistic providing the relevant metric for comparison between individuals in the correlation analysis. The image differences (or summary statistics) are correlated to genotype as noted herein.

Single Photon Emission Computed Tomography (SPECT) is similar to PET and uses gamma ray emitting radioisotopes and a gamma camera to record data that is converted to dimensional images of active brain regions. SPECT relies on an injection of radioactive tracer, which is rapidly taken up by the brain but does not redistribute. These properties of SPECT make it well suited for differential imaging, because it allows for greater patient movement during various tasks. A significant limitation of SPECT is its poor resolution (about 1 cm) compared to that of MRI. SPECT, however, is able to make use of tracers with much longer half-lives than for PET, such as technetium-99, and as a result, is far more widely available (e.g., because an easily accessible cyclotron is not needed to make the relevant isotopes, as is the case for PET). As with fMRI, a summary statistic can be assigned to represent relevant image differences revealed by SPECT scanning, with this statistic providing the relevant metric for comparison between individuals in the correlation analysis. The image differences (or summary statistics) are correlated to genotype as noted herein.

Diffuse Optical Imaging (DOI) or Diffuse Optical Tomography (DOT) is another brain imaging method that uses near infrared light to generate images of the body. The technique measures the optical absorption of hemoglobin, and relies on the absorption spectrum of hemoglobin varying with its oxygenation status. As with fMRI, a summary statistic can be assigned to represent relevant image differences revealed by SPECT scanning, with this statistic providing the relevant metric for comparison between individuals in the correlation analysis. The image differences (or summary statistics) are correlated to genotype as noted herein.

An EEG, or electroencephalograph, is a recording of electrical signals from the brain, made by hooking up electrodes to the subject's scalp. These electrodes pick up electric signals naturally produced by the brain and send them to galvanometers that are in turn hooked up to recording apparatus which record the output from the galvanometer. For purposes of the invention, this output is considered an “image” of the brain, because the EEG recording represents and records brain activity. EEGs permit electrical impulses to be tracked across the surface of the brain in real time. An EEG can show what state a person is in—asleep, awake, anaesthetized—because the characteristic patterns of current differ for each of these states. EEGs can also be used to show how long it takes the brain to process various stimuli. As with fMRI, a summary statistic can be assigned to represent relevant image differences revealed by the EEG, with this statistic providing the relevant metric for comparison between individuals in the correlation analysis. The image differences (or summary statistics) are correlated to genotype as noted herein.

MEG (magnetoencephalography) is a new technology that measures magnetic fields that emanate from the head as a result of brain activity. In MEG, magnetic detection coils bathed in liquid helium are poised over the subject's head. The brain's magnetic field induces a current in the coils, which in turn induces a magnetic field in a superconducting quantum interference device, or SQUID. Of all the brain scanning methods, MEG provides the most accurate resolution of the timing of nerve cell activity. The technology is not yet widely available, due to the cost of the relevant instrumentation, but, regardless, can be used in the context of the present invention. As with fMRI, a summary statistic can be assigned to represent relevant image differences revealed by the MEG, with this statistic providing the relevant metric for comparison between individuals in the correlation analysis. The image differences (or summary statistics) are correlated to genotype as noted herein.

Regardless of which method is used, differences (and differential differences) in scans/images can be determined, e.g., by determining how scans differ from one individual to another, and/or how differences between scanned states differ between individuals. That is, variance can be detected between individuals in a first standardized state (relaxed, asleep, performing a particular cognitive task such as high or low memory, etc), or variance in how individual's scans differ between states can be determined (differences in brain activity between states can differ between individuals).

In either case, a summary statistic can be assigned to represent the difference. This summary statistic can be dimensionless, or can be given dimensions based on the type of scanning technology at issue. For example, a difference in activation between a first and second state for a defined brain region (e.g., as defined by a standard brain atlas such as a Talairach or MNI atlas.

A Talairach atlas (named after French neurosurgeon Jean Talairach) is a coordinate system of the human brain, used to describe the location of brain structures in a manner that is independent of individual differences in the size and overall shape of the brain. This technology is used to spatially warp an individual brain image obtained through MRI, PET, etc. to a standard Talairach space. One disadvantage of the Talairach coordinate atlas is that the atlas was created based on a post-mortem sample from an older woman with a smaller than average cranium. Most individual brains are considerably warped to fit the small size of the atlas, inducing some error in the use of the atlas. Nonetheless, the Talairach atlas is a commonly used tool in modern neuroimaging. A more modern brain atlas is the MNI (Montreal Neurological Institute) atlas. Automated systems for using these atlases to assign neurostructures are available, e.g., Anatomical Automatic Labeling (AAL) is a computer program package that includes a digital human brain atlas. It is particularly used in research-based human functional neuroimaging, where it is used to obtain a neuroanatomical label to a given coordinate in the human brain. This software package is available on the web, e.g., at http://www.cyceron.fr/freeware.

### Overview of Genes Linked to Differential Brain Scan Images

The invention includes new correlations between the genes and any linked loci for the genes of Appendix 1, including, e.g., LOC148823 [C1orf150], PPP1CB, SPDY1, KIAA1604, MGC42174, NPY5R, SFXN1, ARHGAP18, ZNF297B, MKI67, FLJ22531, PC, and SPINL (SPIN1) and a differential brain image phenotype, e.g., associated with a neuropsychiatric disorder.

Neuropsychiatric disorders include disorders comprising neurological and/or psychiatric features such as schizophrenia and other psychotic disorders, bipolar disorder, mood disorders such as major or clinical depression, anxiety disorders such as generalized anxiety disorder, somatoform disorders (Briquet's disorder), factitious disorders such as Munchausen syndrome, dissociative disorders such as dissociative identity disorder, sexual disorders such as dyspareunia and gender identity disorder, eating disorders such as anorexia nervosa, sleep disorders such as insomnia and narcolepsy, impulse control disorders such as kleptomania, adjustment disorders, personality disorders such as narcissistic personality disorder, tardive dyskinesia, tourettes, autism, and many others. Neuropsychiatric disorders also include encompass conditions that can be categorized as “neurodiversity” rather than “illness”, e.g., atypical neurological wiring such as may occur in autism, dyslexia, dyspraxia and hyperactivity.

Certain alleles in, and linked to, these genes or gene products are predictive of the likelihood that an individual possessing the relevant alleles will develop one or more of these disorders. Accordingly, detection of these alleles, by any available method, can be used for diagnostic purposes such as early detection of susceptibility to a disorder, prognosis for patients that present with the disorder, and in assisting diagnosis, e.g., where current criteria are insufficient for a definitive diagnosis.

The identification that the genes of Appendix 1 are correlated to the disorders noted above also provides a platform for screening potential modulators of these disorders. Modulators of the activity of any of these genes or their encoded proteins are expected to have an effect on the disorder that the genes are correlated with. Thus, methods of screening, systems for screening and the like, are features of the invention. Modulators identified by these screening approaches are also a feature of the invention.

Kits for the diagnosis and treatment of these disorders, e.g., comprising probes to identify relevant alleles, modulators, packaging materials, instructions for correlating detection of relevant alleles to neuropsychiatric disorders are also a feature of the invention. These kits can also include modulators of the relevant disorder and/or instructions for treating patients using conventional methods.

### Methods of Identifying Neuropsychiatric Disorders and Related Phenotypes

As noted, the invention provides the discovery that certain genes or other loci (e.g., those of appendix 1, e.g., LOC148823 [C1orf150], PPP1CB, SPDY1, KIAA1604, MGC42174, NPY5R, SFXN1, ARHGAP18, ZNF297B, MKI67, FLJ22531, PC, and SPINL (SPIN1)), are linked to brain scan image phenotype (a differential brain scan phenotype), which is, in turn, linked to a neuropsychiatric disorder such as schizophrenia or bipolar disorder. Thus, by detecting markers (e.g., the SNPs in Appendix 1, or loci closely linked thereto) that correlate, positively or negatively, with the relevant phenotypes, it can be determined whether an individual or population is likely to be susceptible to these phenotypes/disorders.

This ability to use the gene as a proxy for the phenotype or disorder provides enhanced early detection options to identify patients that are likely to eventually suffer from neuropsychiatric disorders, making it possible, in some cases, to prevent actual development of the disorder e.g., by taking early preventative action (e.g., any existing therapy such as available medications, lifestyle modifications (e.g., diet, exercise, stress reduction), psychiatric treatment, etc.). In addition, use of the various markers herein also adds certainty to existing diagnostic techniques for identifying whether a patient is suffering from, e.g., a neuropsychiatric disorder, which can be somewhat ambiguous using previously available methods. Furthermore, knowledge of whether there is a molecular basis for the disorder can also assist in determining patient prognosis, e.g., by providing an indication of how likely it is that a patient can respond to conventional therapy for the relevant disorder, or whether other more serious options such as psychiatric hospitalization are likely to be necessary. Disease treatment can also be specifically targeted based on what type of molecular correlation the patient displays.

In non-human subjects (e.g., non-human mammals such as pets and livestock), it is also possible to similarly use this information both for disease diagnosis and prevention (e.g., treatment of livestock and pets such as dogs and cats, etc.). as in humans. In addition, for such non-human applications, it is also possible to perform marker-assisted animal breeding to eliminate or enhance particular alleles from the population, e.g., to modify behavior predisposition in offspring. In brief, livestock animals or germplasm can be selected for marker alleles that positively or negatively correlate with a disorder, without actually raising the livestock and measuring for the desired trait. Marker assisted selection (MAS) is a powerful shortcut to selecting for desired phenotypes and for introgressing desired traits into livestock or pet groups (e.g., introgressing desired traits into elite herd or other breeding populations). MAS is easily adapted to high throughput molecular analysis methods that can quickly screen genetic material for the markers of interest, and is much more cost effective than raising and observing livestock for observable traits.

Detection methods for detecting relevant alleles can include any available method, e.g., amplification technologies. For example, detection can include amplifying the polymorphism or a sequence associated therewith and detecting the resulting amplicon. This can include admixing an amplification primer or amplification primer pair with a nucleic acid template isolated from the organism or biological sample (e.g., comprising the SNP or other polymorphism), e.g., where the primer or primer pair is complementary or partially complementary to at least a portion of the gene or tightly linked polymorphism, or to a sequence proximal thereto. The primer is typically capable of initiating nucleic acid polymerization by a polymerase on the nucleic acid template. The primer or primer pair is extended, e.g., in a DNA polymerization reaction (PCR, RT-PCR, etc.) comprising a polymerase and the template nucleic acid to generate the amplicon. The amplicon is detected by any available detection process, e.g., sequencing, hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like.

The correlation between a detected polymorphism and a trait can be performed by any method that can identify a relationship between an allele and a phenotype. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Within the context of these methods, the following discussion first focuses on how markers and alleles are linked and how this phenomenon can be used in the context of methods for identifying neuropsychiatric disorders, and then focuses on marker detection methods.

Markers, Linkage and Alleles

In traditional linkage (or association) analysis, no direct knowledge of the physical relationship of genes on a chromosome is required. Mendel's first law is that factors of pairs of characters are segregated, meaning that alleles of a diploid trait separate into two gametes and then into different offspring. Classical linkage analysis can be thought of as a statistical description of the relative frequencies of cosegregation of different traits. Linkage analysis is the well characterized descriptive framework of how traits are grouped together based upon the frequency with which they segregate together.

That is, if two non-allelic traits are inherited together with a greater than random frequency, they are said to be “linked.” The frequency with which the traits are inherited together is the primary measure of how tightly the traits are linked, i.e., traits which are inherited together with a higher frequency are more closely linked than traits which are inherited together with lower (but still above random) frequency. Traits are linked because the genes which underlie the traits reside near one another on the same chromosome. The further apart on a chromosome the genes reside, the less likely they are to segregate together, because homologous chromosomes recombine during meiosis. Thus, the further apart on a chromosome the genes reside, the more likely it is that there will be a recombination event during meiosis that will result in two genes segregating separately into progeny.

A common measure of linkage (or association) is the frequency with which traits cosegregate. This can be expressed as a percentage of cosegregation (recombination frequency) or, also commonly, in centiMorgans (cM), which are actually a reciprocal unit of recombination frequency. The cM is named after the pioneering geneticist Thomas Hunt Morgan and is a unit of measure of genetic recombination frequency. One cM is equal to a 1% chance that a trait at one genetic locus will be separated from a trait at another locus due to recombination in a single generation (meaning the traits segregate together 99% of the time). Because chromosomal distance is approximately proportional to the frequency of recombination events between traits, there is an approximate physical distance that correlates with recombination frequency. For example, in humans, 1 cM correlates, on average, to about 1 million base pairs (1 Mbp).

Marker loci are themselves traits and can be assessed according to standard linkage analysis by tracking the marker loci during segregation. Thus, in the context of the present invention, one cM is equal to a 1% chance that a marker locus will be separated from another locus (which can be any other trait, e.g., another marker locus, or another trait locus that encodes a QTL for the phenotype or disorder of interest), due to recombination in a single generation. The markers herein, e.g., those listed in Appendix 1 (or that can be derived from the information in Appendix 1), can correlate with neuropsychiatric disorders. This means that the markers comprise or are sufficiently proximal to a QTL for the disorder (or related phenotype, such as a disorder-dependent differential bran image) that they can be used as a predictor for the trait (disorder/image) itself. This is extremely useful in the context of disease diagnosis and, in livestock applications, for marker assisted selection (MAS).

From the foregoing, it is clear that any marker that is linked to a trait locus of interest (e.g., in the present case, a QTL or identified linked marker locus for the neuropsychiatric disorder/brain image phenotype, e.g., as in Appendix 1) can be used as a marker for that trait. Thus, in addition to the markers noted in Appendix 1, other markers closely linked to the markers itemized in Appendix 1 can also usefully predict the presence of the marker alleles indicated in Appendix 1 (and, thus, the relevant trait). Such linked markers are particularly useful when they are sufficiently proximal to a given locus so that they display a low recombination frequency with the given locus. In the present invention, such closely linked markers are a feature of the invention. Closely linked loci display a recombination frequency with a given marker of about 20% or less (the given marker is within 20 cM of the given marker). Put another way, closely linked loci co-segregate at least 80% of the time. More preferably, the recombination frequency is 10% or less, e.g., 9%, 8%, 7%, 6%, 5%, 4%, 3%, 2%, 1%, 0.5%, 0.25%, or 0.1% or less. In one typical class of embodiments, closely linked loci are within 5 cM or less of each other.

As one of skill in the art will recognize, recombination frequencies (and, as a result, map positions) can vary depending on the map used (and the markers that are on the map). Additional markers that are closely linked to (e.g., within about 10 cM, or more preferably within about 1 cM of) the markers identified in Appendix 1 may readily be used for identification of QTL for a neuropsychiatric disorder.

Marker loci are especially useful in the present invention when they are closely linked to target loci (e.g., QTL for a disorder of interest), or, alternatively, simply other marker loci, such as those itemized in Appendix 1 that are, themselves linked to such QTL that they are being used as markers for. The more closely a marker is linked to a target locus that encodes or affects a phenotypic trait, the better an indicator for the target locus that the marker is (due to the reduced cross-over frequency between the target locus and the marker). Thus, in one embodiment, closely linked loci such as a marker locus and a second locus (e.g., a given marker locus of Appendix 1 and an additional second locus) display an inter-locus cross-over frequency of about 20% or less, e.g., 15% or less, preferably 10% or less, more preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus such as a QTL) display a recombination a frequency of about 1% or less, e.g., about 0.75% or less, more preferably about 0.5% or less, or yet more preferably about 0.25% or 0.1% or less. Thus, the loci are about 20 cM, 19 cM, 18 cM, 17 cM, 16 cM, 15 cM, 14 cM, 13 cM, 12 cM, 11 cM, 10 cM, 9 cM, 8 cM, 7 cM, 6 cM, 5 cM, 4 cM, 3 cM, 2 cM, 1 cM, 0.75 cM, 0.5 cM, 0.25 cM, 0 or 0.1 cM or less apart. Put another way, two loci that are localized to the same chromosome, and at such a distance that recombination between the two loci occurs at a frequency of less than 20% (e.g., about 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25%, 0.1% or less) are said to be “proximal to” each other. In one aspect, linked markers are within 100 kb (which correlates in humans to about 0.1 cM, depending on local recombination rate), e.g., 50 kb, or even 20 kb or less of each other. It is worth noting that the entire human genome is available, and millions of polymorphisms in the human genome are also known, making it possible for one of skill to routinely select markers that lie proximal to essentially any given marker or QTL.

When referring to the relationship between two genetic elements, such as a genetic element contributing to a neuropsychiatric disorder, and a proximal marker, “coupling” phase linkage indicates the state where the “favorable” allele at the locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for the disorder) is physically linked with an “unfavorable” allele at the proximal marker locus, and the two “favorable” alleles are not inherited together (i.e., the two loci are “out of phase” with each other).

In addition to tracking SNP and other polymorphisms in the genome, and in corresponding expressed nucleic acids and polypeptides, expression level differences between individuals or populations for the products of the genes of Appendix 1, in either mRNA or protein form, can also correlate to the disorder. Accordingly, markers of the invention can include any of, e.g.: genomic loci, transcribed nucleic acids, spliced nucleic acids, expressed proteins, levels of transcribed nucleic acids, levels of spliced nucleic acids, and levels of expressed proteins.

Marker Amplification Strategies

Amplification primers for amplifying markers (e.g., marker loci) and suitable probes to detect such markers or to genotype a sample with respect to multiple marker alleles, are a feature of the invention. In Appendix 1, specific loci for amplification are provided, (optionally in conjunction with known flanking sequences) for the design of such primers. Also, there are publicly available programs such as “Oligo” available for primer design. With such available primer selection and design software, the publicly available human genome sequence and the polymorphism locations as provided in Appendix 1, one of skill can routinely design primers to amplify the SNPs of the present invention. Further, it will be appreciated that the precise probe to be used for detection of a nucleic acid comprising a SNP (e.g., an amplicon comprising the SNP) can vary, e.g., any probe that can identify the region of a marker amplicon to be detected can be used in conjunction with the present invention. Further, the configuration of the detection probes can, of course, vary. Thus, the invention is not limited to the sequences recited herein.

Indeed, it will be appreciated that amplification is not a requirement for marker detection—for example, one can directly detect unamplified genomic DNA simply by performing a Southern blot on a sample of genomic DNA, or by using available “branched DNA” (bDNA) probe technologies (available, e.g., from Panomics, Inc. Hayward, Calif.). Procedures for performing Southern blotting, standard amplification (PCR, LCR, or the like) and many other nucleic acid detection methods are well established and are taught, e.g., in Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis et al. eds) Academic Press Inc. San Diego, Calif. (1990) (Innis).

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Typically, molecular markers are detected by any established method available in the art, including, without limitation, allele specific hybridization (ASH), detection of single nucleotide extension, array hybridization (optionally including ASH), or other methods for detecting single nucleotide polymorphisms (SNPs), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme marker detection, northern analysis (where expression levels are used as markers), quantitative amplification of mRNA or cDNA, or the like. While the exemplary markers provided in the appendix herein are SNP markers, any of the aforementioned marker types can be employed in the context of the invention to identify linked loci that affect or effect a neuropsychiatric disorder or brain image phenotype.

Example Techniques for Marker Detection

The invention provides molecular markers that comprise or are linked to QTL for the disorders or phenotypes herein. The markers find use in disease predisposition diagnosis, prognosis, treatment and for marker assisted selection for desired traits in livestock/pets. It is not intended that the invention be limited to any particular method for the detection of these markers.

Markers corresponding to genetic polymorphisms between members of a population can be detected by numerous methods well-established in the art (e.g., PCR-based sequence specific amplification, restriction fragment length polymorphisms (RFLPs), isozyme markers, northern analysis, allele specific hybridization (ASH), array based hybridization, amplified variable sequences of the genome, self-sustained sequence replication, simple sequence repeat (SSR), single nucleotide polymorphism (SNP), random amplified polymorphic DNA (“RAPD”) or amplified fragment length polymorphisms (AFLP). In one additional embodiment, the presence or absence of a molecular marker is determined simply through nucleotide sequencing of the polymorphic marker region. Any of these methods are readily adapted to high throughput analysis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, N.Y., as well as in Sambrook, Berger and Ausubel.

For example, markers that comprise restriction fragment length polymorphisms (RFLP) are detected, e.g., by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals or populations. Determining one or more restriction enzyme that produces informative fragments for each allele of a marker is a simple procedure, well known in the art. After separation by length in an appropriate matrix (e.g., agarose or polyacrylamide) and transfer to a membrane (e.g., nitrocellulose, nylon, etc.), the labeled probe is hybridized under conditions which result in equilibrium binding of the probe to the target followed by removal of excess probe by washing.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes, and colorimetric labels. Other labels include ligands that bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (2003) Handbook of Fluorescent Probes and Research Chemicals Ninth Edition by Molecular Probes, Inc. (Eugene Oreg.). Additional details regarding marker detection strategies are found below.

Amplification-Based Detection Methods

PCR, RT-PCR and LCR are in particularly broad use as amplification and amplification-detection methods for amplifying nucleic acids of interest (e.g., those comprising marker loci), facilitating detection of the nucleic acids of interest. Details regarding the use of these and other amplification methods can be found in any of a variety of standard texts, including, e.g., Sambrook, Ausubel, and Berger. Many available biology texts also have extended discussions regarding PCR and related amplification methods. One of skill will appreciate that essentially any RNA can be converted into a double stranded DNA suitable for restriction digestion, PCR expansion and sequencing using reverse transcriptase and a polymerase (“Reverse Transcription-PCR, or “RT-PCR”). See also, Ausubel, Sambrook and Berger, above. These methods can also be used to quantitatively amplify mRNA or corresponding cDNA, providing an indication of expression levels of mRNA that correspond to the genes or gene products of Appendix 1 in an individual. Differences in expression levels for these genes between individuals, families, lines and/or populations can also be used as markers for a neuropsychiatric disorder.

Real Time Amplification/Detection Methods

In one aspect, real time PCR or LCR is performed on the amplification mixtures described herein, e.g., using molecular beacons or TaqMan™ probes. A molecular beacon (MB) is an oligonucleotide or PNA which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature and MBs are available from a number of commercial reagent sources. See also, e.g., Leone et al. (1995) “Molecular beacon probes combined with amplification by NASBA enable homogenous real-time detection of RNA.” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. No. 5,925,517 (Jul. 20, 1999) to Tyagi et al. entitled “Detectably labeled dual conformation oligonucleotide probes, assays and kits;” U.S. Pat. No. 6,150,097 to Tyagi et al (Nov. 21, 2000) entitled “Nucleic acid detection probes having non-FRET fluorescence quenching and kits and assays including such probes” and U.S. Pat. No. 6,037,130 to Tyagi et al (Mar. 14, 2000), entitled “Wavelength-shifting probes and primers and their use in assays and kits.”

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO92/02638.

Other similar methods include e.g. fluorescence resonance energy transfer between two adjacently hybridized probes, e.g., using the “LightCycler®” format described in U.S. Pat. No. 6,174,670.

Array-Based Marker Detection

Array-based detection can be performed using commercially available arrays, e.g., from Affymetrix (Santa Clara, Calif.). Perlegen (Santa Clara, Calif.), or other manufacturers. Reviews regarding the operation of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Chee et al. (1996) “Accessing Genetic Information with High-Density DNA Arrays.” Science 274:610-614. Array based detection is a preferred method for identification markers of the invention in samples, due to the inherently high-throughput nature of array based detection. In addition, relationships between different genes and phenotypes can be simultaneously assessed in a single assay using these methods.

A variety of probe arrays have been described in the literature and can be used in the context of the present invention for detection of markers that can be correlated to the phenotypes/disorders noted herein. For example, DNA probe array chips or larger DNA probe array wafers (from which individual chips would otherwise be obtained by breaking up the wafer) are used in one embodiment of the invention. DNA probe array wafers generally comprise glass wafers on which high density arrays of DNA probes (short segments of DNA) have been placed. Each of these wafers can hold, for example, approximately 60 million DNA probes that are used to recognize longer sample DNA sequences (e.g., from individuals or populations, e.g., that comprise markers of interest). The recognition of sample DNA by the set of DNA probes on the glass wafer takes place through DNA hybridization. When a DNA sample hybridizes with an array of DNA probes, the sample binds to those probes that are complementary to the sample DNA sequence. By evaluating to which probes the sample DNA for an individual hybridizes more strongly, it is possible to determine whether a known sequence of nucleic acid is present or not in the sample, thereby determining whether a marker found in the nucleic acid is present. One can also use this approach to perform ASH, by controlling the hybridization conditions to permit single nucleotide discrimination, e.g., for SNP identification and for genotyping a sample for one or more SNPs.

The use of DNA probe arrays to obtain allele information typically involves the following general steps: design and manufacture of DNA probe arrays, preparation of the sample, hybridization of sample DNA to the array, detection of hybridization events and data analysis to determine sequence. Preferred wafers are manufactured using a process adapted from semiconductor manufacturing to achieve cost effectiveness and high quality, and are available, e.g., from Affymetrix, Inc of Santa Clara, Calif.

For example, probe arrays can be manufactured by light-directed chemical synthesis processes, which combine solid-phase chemical synthesis with photolithographic fabrication techniques as employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays can be synthesized simultaneously on a large glass wafer. This parallel process enhances reproducibility and helps achieve economies of scale.

Once fabricated, DNA probe arrays can be used to obtain data regarding presence and/or expression levels for markers of interest. The DNA samples may be tagged with biotin and/or a fluorescent reporter group by standard biochemical methods. The labeled samples are incubated with an array, and segments of the samples bind, or hybridize, with complementary sequences on the array. The array can be washed and/or stained to produce a hybridization pattern. The array is then scanned and the patterns of hybridization are detected by emission of light from the fluorescent reporter groups. Additional details regarding these procedures are found in the examples below. Because the identity and position of each probe on the array is known, the nature of the DNA sequences in the sample applied to the array can be determined. When these arrays are used for genotyping experiments, they can be referred to as genotyping arrays.

The nucleic acid sample to be analyzed is isolated, amplified and, typically, labeled with biotin and/or a fluorescent reporter group. The labeled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labeled nucleic acid, which is now bound to the probe array. Probes that most clearly match the labeled nucleic acid produce stronger signals than those that have mismatches. Since the sequence and position of each probe on the array are known, by complementarity, the identity of the nucleic acid sample applied to the probe array can be identified.

In one embodiment, two DNA samples may be differentially labeled and hybridized with a single set of the designed genotyping arrays. In this way two sets of data can be obtained from the same physical arrays. Labels that can be used include, but are not limited to, cychrome, fluorescein, or biotin (later stained with phycoerythrin-streptavidin after hybridization). Two-color labeling is described in U.S. Pat. No. 6,342,355, incorporated herein by reference in its entirety. Each array may be scanned such that the signal from both labels is detected simultaneously, or may be scanned twice to detect each signal separately.

Intensity data is collected by the scanner for all the markers for each of the individuals that are tested for presence of the marker. The measured intensities are a measure indicative of the amount of a particular marker present in the sample for a given individual (expression level and/or number of copies of the allele present in an individual, depending on whether genomic or expressed nucleic acids are analyzed). This can be used to determine whether the individual is homozygous or heterozygous for the marker of interest. The intensity data is processed to provide corresponding marker information for the various intensities.

Additional Details Regarding Amplified Variable Sequences, SSR, AFLP ASH, SNPs and Isozyme Markers

Amplified variable sequences refer to amplified sequences of the genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the genome serves as a template for amplification with primers that flank a variable sequence of DNA. The variable sequence is amplified and then sequenced.

Alternatively, self-sustained sequence replication can be used to identify genetic markers. Self-sustained sequence replication refers to a method of nucleic acid amplification using target nucleic acid sequences which are replicated exponentially, in vitro, under substantially isothermal conditions by using three enzymatic activities involved in retroviral replication: (1) reverse transcriptase, (2) Rnase H, and (3) a DNA-dependent RNA polymerase (Guatelli et al. (1990) Proc Natl Acad Sci USA 87:1874). By mimicking the retroviral strategy of RNA replication by means of cDNA intermediates, this reaction accumulates cDNA and RNA copies of the original target.

Amplified fragment length polymorphisms (AFLP) can also be used as genetic markers (Vos et al. (1995) Nucl Acids Res 23:4407). The phrase “amplified fragment length polymorphism” refers to selected restriction fragments which are amplified before or after cleavage by a restriction endonuclease. The amplification step allows easier detection of specific restriction fragments. AFLP allows the detection large numbers of polymorphic markers and has been used for genetic mapping (Becker et al. (1995) Mol Gen Genet 249:65; and Meksem et al. (1995) Mol Gen Genet 249:74).

Allele-specific hybridization (ASH) can be used to identify the genetic markers of the invention. ASH technology is based on the stable annealing of a short, single-stranded, oligonucleotide probe to a completely complementary single-strand target nucleic acid. Detection may be accomplished via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described in U.S. Pat. No. 5,468,613, the ASH probe sequence may be bound to a membrane.

In one embodiment, ASH data are typically obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on e.g., an acrylamide gel. However, alternative modes of detection, such as hybridization, e.g., ASH, or RFLP analysis are also appropriate.

Isozyme markers can be employed as genetic markers, e.g., to track isozyme markers linked to the markers herein. Isozymes are multiple forms of enzymes that differ from one another in their amino acid, and therefore their nucleic acid sequences. Some isozymes are multimeric enzymes contain slightly different subunits. Other isozymes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

Additional Details Regarding Nucleic Acid Amplification

As noted, nucleic acid amplification techniques such as PCR and LCR are well known in the art and can be applied to the present invention to amplify and/or detect nucleic acids of interest, such as nucleic acids comprising marker loci. Examples of techniques sufficient to direct persons of skill through such in vitro methods, including the polymerase chain reaction (PCR), the ligase chain reaction (LCR), Qβ-replicase amplification and other RNA polymerase mediated techniques (e.g., NASBA), are found in the references noted above, e.g., Innis, Sambrook, Ausubel, and Berger. Additional details are found in Mullis et al. (1987) U.S. Pat. No. 4,683,202; Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of amplifying large nucleic acids by PCR, which is useful in the context of positional cloning, are further summarized in Cheng et al. (1994) Nature 369: 684, and the references therein, in which PCR amplicons of up to 40 kb are generated. Methods for long-range PCR are disclosed, for example, in U.S. Pat. No. 6,740,510, issued May 25, 2004, entitled “Methods for Amplification of Nucleic Acids”.

Detection of Protein Expression Products

Proteins such as those encoded by the genes in Appendix 1 are encoded by nucleic acids, including those comprising markers that are correlated to the phenotypes of interest herein. For a description of the basic paradigm of molecular biology, including the expression (transcription and/or translation) of DNA into RNA into protein, see, Alberts et al. (2002) Molecular Biology of the Cell, 4^{th} Edition Taylor and Francis, Inc., ISBN: 0815332181 (“Alberts”), and Lodish et al. (1999) Molecular Cell Biology, 4^{th} Edition W H Freeman & Co, ISBN: 071673706X (“Lodish”). Accordingly, proteins corresponding to genes in Appendix 1 can be detected as markers, e.g., by detecting different protein isotypes between individuals or populations, or by detecting a differential presence, absence or expression level of such a protein of interest (e.g., expression level of a gene product of Appendix 1).

A variety of protein detection methods are known and can be used to distinguish markers. In addition to the various references noted supra, a variety of protein manipulation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

“Proteomic” detection methods, which detect many proteins simultaneously have been described. These can include various multidimensional electrophoresis methods (e.g., 2-d gel electrophoresis), mass spectrometry based methods (e.g., SELDI, MALDI, electrospray, etc.), or surface plasmon resonance methods. For example, in MALDI, a sample is usually mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp). Similarly, for SELDI, a first aliquot is contacted with a solid support-bound (e.g., substrate-bound) adsorbent. A substrate is typically a probe (e.g., a biochip) that can be positioned in an interrogatable relationship with a gas phase ion spectrometer. SELDI is also a well known technique, and has been applied to diagnostic proteomics. See, e.g. Issaq et al. (2003) “SELDI-TOF MS for Diagnostic Proteomics” Analytical Chemistry 75:149 A-155A.

In general, the above methods can be used to detect different forms (alleles) of proteins and/or can be used to detect different expression levels of the proteins (which can be due to allelic differences) between individuals, families, lines, populations, etc. Differences in expression levels, when controlled for environmental factors, can be indicative of different alleles at a QTL for the gene of interest, even if the encoded differentially expressed proteins are themselves identical. This occurs, for example, where there are multiple allelic forms of a gene in non-coding regions, e.g., regions such as promoters or enhancers that control gene expression. Thus, detection of differential expression levels can be used as a method of detecting allelic differences.

In other aspect of the present invention, a gene comprising, in linkage disequilibrium with, or under the control of a nucleic acid associated with a disorder or phenotype herein may exhibit differential allelic expression. “Differential allelic expression” as used herein refers to both qualitative and quantitative differences in the allelic expression of multiple alleles of a single gene present in a cell. As such, a gene displaying differential allelic expression may have one allele expressed at a different time or level as compared to a second allele in the same cell/tissue. For example, an allele associated with a neuropsychiatric disorder may, in some cases, be expressed at a higher or lower level than an allele that is not associated with the disorder, even though both are alleles of the same gene and are present in the same cell/tissue.

Additional Details Regarding Types of Markers Appropriate for Screening

The biological markers that are screened for correlation to the phenotypes herein can be any of those types of markers that can be detected by screening, e.g., genetic markers such as allelic variants of a genetic locus (e.g., as in SNPs), expression markers (e.g., presence or quantity of mRNAs and/or proteins), and/or the like.

The nucleic acid of interest to be amplified, transcribed, translated and/or detected in the methods of the invention can be essentially any nucleic acid, though nucleic acids derived from human sources are especially relevant to the detection of markers associated with disease diagnosis and clinical applications. The sequences for many nucleic acids and amino acids (from which nucleic acid sequences can be derived via reverse translation) are available, including for the genes or gene products of Appendix 1. Common sequence repositories for known nucleic acids include GenBank® EMBL, DDBJ and the NCBI. Other repositories can easily be identified by searching the internet. The nucleic acid to be amplified, transcribed, translated and/or detected can be an RNA (e.g., where amplification includes RT-PCR or LCR, the Van-Gelder Eberwine reaction or Ribo-SPIA) or DNA (e.g., amplified DNA, cDNA or genomic DNA), or even any analogue thereof (e.g., for detection of synthetic nucleic acids or analogues thereof, e.g., where the sample of interest includes or is used to derive or synthesize artificial nucleic acids). Any variation in a nucleic acid sequence or expression level between individuals or populations can be detected as a marker, e.g., a mutation, a polymorphism, a single nucleotide polymorphism (SNP), an allele, an isotype, expression of an RNA or protein, etc. One can detect variation in sequence, expression levels or gene copy numbers as markers that can be correlated, e.g., to a differential bran image or neuropsychiatric disorder.

For example, the methods of the invention are useful in screening samples derived from patients for a marker nucleic acid of interest, e.g., from bodily fluids (blood, saliva, urine etc.), tissue, and/or waste from the patient. Thus, stool, sputum, saliva, blood, lymph, tears, sweat, urine, vaginal secretions, ejaculatory fluid or the like can easily be screened for nucleic acids by the methods of the invention, as can essentially any tissue of interest that contains the appropriate nucleic acids. These samples are typically taken, following informed consent, from a patient by standard medical laboratory methods.

Prior to amplification and/or detection of a nucleic acid comprising a marker, the nucleic acid is optionally purified from the samples by any available method, e.g., those taught in Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”); and/or Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). A plethora of kits are also commercially available for the purification of nucleic acids from cells or other samples (see, e.g., EasyPrep™, FlexiPrep™, both from Pharmacia Biotech; StrataClean™, from Stratagene; and, QIAprep™ from Qiagen). Alternately, samples can simply be directly subjected to amplification or detection, e.g., following aliquotting and/or dilution.

Examples of markers can include polymorphisms, single nucleotide polymorphisms, microsatellite markers, presence of one or more nucleic acids in a sample, absence of one or more nucleic acids in a sample, presence of one or more genomic DNA sequences, absence or one or more genomic DNA sequences, presence of one or more mRNAs, absence of one or more mRNAs, expression levels of one or more mRNAs, presence of one or more proteins, expression levels of one or more proteins, and/or data derived from any of the preceding or combinations thereof. Essentially any number of markers can be detected, using available methods, e.g., using array technologies that provide high density, high throughput marker mapping. Thus, at least about 10, 100, 1,000, 10,000, or even 100,000 or more genetic markers can be tested, simultaneously or in a serial fashion (or combination thereof), for correlation to a relevant phenotype, in the first and/or second population. Combinations of markers can also be desirably tested, e.g., to identify genetic combinations or combinations of expression patterns in populations that are correlated to the phenotype.

As noted, the biological marker to be detected can be any detectable biological component. Commonly detected markers include genetic markers (e.g., DNA sequence markers present in genomic DNA or expression products thereof) and expression markers (which can reflect genetically coded factors, environmental factors, or both). Where the markers are expression markers, the methods can include determining a first expression profile for a first individual or population (e.g., of one or more expressed markers, e.g., a set of expressed markers) and comparing the first expression profile to a second expression profile for the second individual or population. In this example, correlating expression marker(s) to a particular phenotype can include correlating the first or second expression profile to the phenotype of interest.

Probe/Primer Synthesis Methods

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others. Similarly, PNAs can be custom ordered from any of a variety of sources, such as PeptidoGenic (pkim@ccnet.com), HTI Bio-products, inc. (htibio.com), BMA Biomedicals Ltd (U.K.), Bio Synthesis, Inc., and many others.

In Silico Marker Detection

In some embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors. The entire human genome has been sequenced and, thus, sequence information can be used to identify marker regions, flanking nucleic acids, etc.

Amplification Primers for Marker Detection

In some preferred embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region.

It will be appreciated that, although many specific examples of primers are provided herein (see, Appendix 1), suitable primers to be used with the invention can be designed using any suitable method. It is not intended that the invention be limited to any particular primer or primer pair. For example, primers can be designed using any suitable software program, such as LASERGENE®, e.g., taking account of publicly available sequence information.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers of the invention be limited to generating an amplicon of any particular size. For example, primers used to amplify the marker loci and alleles herein are not limited to amplifying the entire region of the relevant locus. The primers can generate an amplicon of any suitable length that is longer or shorter than any given example amplicon. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

Detection of Markers for Positional Cloning

In some embodiments, a nucleic acid probe is used to detect a nucleic acid that comprises a marker sequence. Such probes can be used, for example, in positional cloning to isolate nucleotide sequences linked to the marker nucleotide sequence. It is not intended that the nucleic acid probes of the invention be limited to any particular size. In some embodiments, nucleic acid probe is at least 20 nucleotides in length, or alternatively, at least 50 nucleotides in length, or alternatively, at least 100 nucleotides in length, or alternatively, at least 200 nucleotides in length.

A hybridized probe is detected using, autoradiography, fluorography or other similar detection techniques depending on the label to be detected. Examples of specific hybridization protocols are widely available in the art, see, e.g., Berger, Sambrook, and Ausubel, all herein.

### Generation of Transgenic Cells and Organisms

The present invention also provides cells and organisms which are transformed with nucleic acids corresponding to QTL identified according to the invention. For example, such nucleic acids include chromosome intervals (e.g., genomic fragments), ORFs and/or cDNAs that encode genes that correspond or are linked to QTL for neuropsychiatric disorders or related phenotypes (e.g., differential brain scans). Additionally, the invention provides for the production of polypeptides or nucleic acids (e.g., anti-sense, RNAi, etc.) that influence these disorders/phenotypes. This is useful, e.g., to influence treatment of the disorders, and to study the disorders/phenotypes, e.g., in animal models.

The generation of transgenic cells also provides commercially useful cells having defined genes that influence the relevant phenotype, thereby providing a platform for screening potential modulators of the phenotype, as well as basic research into the mechanism of action for each of the genes of interest. In addition, gene therapy can be used to introduce desirable genes into individuals or populations thereof, or to controllably inhibit expression (e.g., using RNAi, antisense, or the like). Such gene therapies may be used to provide a treatment for a disorder exhibited by an individual, or may be used as a preventative measure to prevent the development of such a disorder in an individual at risk.

Knock-out animals, such as knock-out mice, can be produced for any of the genes noted herein, to further identify phenotypic effects of the genes. Similarly, recombinant mice or other animals can be used as models for human disease, e.g., by knocking out any natural gene herein and introduction (e.g., via homologous recombination) of the human (or other species) gene into the animal. The effects of modulators on the heterologous human genes and gene products can then be monitored in the resulting in vivo model animal system.

General texts which describe molecular biological techniques for the cloning and manipulation of nucleic acids and production of encoded polypeptides include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2001 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2004 or later) (“Ausubel”)). These texts describe mutagenesis, the use of vectors, promoters and many other relevant topics related to, e.g., the generation of clones that comprise nucleic acids of interest, e.g., genes, marker loci, marker probes, QTL that segregate with marker loci, etc.

Host cells are genetically engineered (e.g., transduced, transfected, transformed, etc.) with the vectors of this invention (e.g., vectors, such as expression vectors which comprise an ORF derived from or related to a QTL) which can be, for example, a cloning vector, a shuttle vector or an expression vector. Such vectors are, for example, in the form of a plasmid, a phagemid, an agrobacterium, a virus, a naked polynucleotide (linear or circular), or a conjugated polynucleotide. Vectors can be introduced into bacteria, especially for the purpose of propagation and expansion. Additional details regarding nucleic acid introduction methods are found in Sambrook, Berger and Ausubel, infra. The method of introducing a nucleic acid of the present invention into a host cell is not critical to the instant invention, and it is not intended that the invention be limited to any particular method for introducing exogenous genetic material into a host cell. Thus, any suitable method, e.g., including but not limited to the methods provided herein, which provides for effective introduction of a nucleic acid into a cell or protoplast can be employed and finds use with the invention.

The engineered host cells can be cultured in conventional nutrient media modified as appropriate for such activities as, for example, activating promoters or selecting transformants. In addition to Sambrook, Berger and Ausubel, all infra, Atlas and Parks (eds) The Handbook of Microbiological Media (1993) CRC Press, Boca Raton, Fla. and available commercial literature such as the Life Science Research Cell Culture Catalogue (2004) from Sigma-Aldrich, Inc (St Louis, Mo.) (“Sigma-LSRCCC”) provide additional details.

Making Knock-Out Animals and Transgenics

Transgenic animals are a useful tool for studying gene function and testing putative gene or gene product modulators. Human (or other selected species) genes herein can be introduced in place of endogenous genes of a laboratory animal, making it possible to study function of the human (or other, e.g., livestock) gene or gene product in the easily manipulated and studied laboratory animal.

It will be appreciated that there is not always a precise correspondence for responses to modulators between homologous gene in different animals, making the ability to study the human or other species of interest (e.g., a livestock species) in a laboratory animal particularly useful. Although similar genetic manipulations can be performed in tissue culture, the interaction of genes and gene products in the context of an intact organism provides a more complete and physiologically relevant picture of such genes and gene products than can be achieved in simple cell-based screening assays. This is particularly useful in the present invention, where complex interactions in the brain may ultimately be at issue. Accordingly, one feature of the invention is the creation of transgenic animals comprising heterologous genes of interest, e.g., a heterologous gene from Appendix 1.

In general, such a transgenic animal is simply an animal that has had appropriate genes (or partial genes, e.g., comprising coding sequences coupled to a promoter) introduced into one or more of its cells artificially. This is most commonly done in one of two ways. First, a DNA can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing the (heterologous) DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (random insertion).

One common use of targeted insertion of DNA is to make knock-out mice. Typically, homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Gene expression can also be inhibited by RNA silencing or interference. “RNA silencing” refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA).

### Inhibiting Expression by RNAI/Antisense

As noted, there are several applications for inhibiting gene expression of one or more of the genes in Appendix 1. These include therapeutic applications and also include inhibition in animal models (including transgenic animal models noted above). The most common ways of inhibiting expression are to use either antisense or RNAi based technologies.

For example, use of antisense nucleic acids is well known in the art. An antisense nucleic acid has a region of complementarity to a target nucleic acid, e.g., an mRNA or DNA corresponding to a gene of Appendix 1. Typically, a nucleic acid comprising a nucleotide sequence in a complementary, antisense orientation with respect to a coding (sense) sequence of an endogenous gene is introduced into a cell. The antisense nucleic acid can be RNA, DNA, a PNA or any other appropriate molecule. A duplex can form between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced, e.g., for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, e.g., in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al (1991) Symp Soc. Exp Biol 45:117-127; de Lange et al., (1995) Curr Top Microbiol Immunol 197:57-75; Hamilton et al. (1995) Curr Top Microbiol Immunol 197:77-89; Finnegan et al., (1996) Proc Natl Acad Sci USA 93:8449-8454; Uhlmann and A. Pepan (1990), Chem. Rev. 90:543; P. D. Cook (1991), Anti-Cancer Drug Design 6:585; J. Goodchild, Bioconjugate Chem. 1 (1990) 165; and, S. L. Beaucage and R. P. Iyer (1993), Tetrahedron 49:6123; and F. Eckstein, Ed. (1991), Oligonucleotides and Analogues—A Practical Approach, IRL Press.

Gene expression can also be inhibited by RNA silencing or interference. “RNA silencing” refers to any mechanism through which the presence of a single-stranded or, typically, a double-stranded RNA in a cell results in inhibition of expression of a target gene comprising a sequence identical or nearly identical to that of the RNA, including, but not limited to, RNA interference, repression of translation of a target mRNA transcribed from the target gene without alteration of the mRNA's stability, and transcriptional silencing (e.g., histone acetylation and heterochromatin formation leading to inhibition of transcription of the target mRNA).

The term “RNA interference” (“RNAi,” sometimes called RNA-mediated interference, post-transcriptional gene silencing, or quelling) refers to a phenomenon in which the presence of RNA, typically double-stranded RNA, in a cell results in inhibition of expression of a gene comprising a sequence identical, or nearly identical, to that of the double-stranded RNA. The double-stranded RNA responsible for inducing RNAi is called an “interfering RNA.” Expression of the gene is inhibited by the mechanism of RNAi as described below, in which the presence of the interfering RNA results in degradation of mRNA transcribed from the gene and thus in decreased levels of the mRNA and any encoded protein.

The mechanism of RNAi has been and is being extensively investigated in a number of eukaryotic organisms and cell types. See, for example, the following reviews: McManus and Sharp (2002) “Gene silencing in mammals by small interfering RNAs” Nature Reviews Genetics 3:737-747; Hutvagner and Zamore (2002) “RNAi: Nature abhors a double strand” Curr Opin Genet & Dev 200:225-232; Hannon (2002) “RNA interference” Nature 418:244-251; Agami (2002) “RNAi and related mechanisms and their potential use for therapy” Curr Opin Chem Biol 6:829-834; Tuschl and Borkhardt (2002) “Small interfering RNAs: A revolutionary tool for the analysis of gene function and gene therapy” Molecular Interventions 2:158-167; Nishikura (2001) “A short primer on RNAi: RNA-directed RNA polymerase acts as a key catalyst” Cell 107:415-418; and Zamore (2001) “RNA interference: Listening to the sound of silence” Nature Structural Biology 8:746-750. RNAi is also described in the patent literature; see, e.g., CA 2359180 by Kreutzer and Limmer entitled “Method and medicament for inhibiting the expression of a given gene”; WO 01/68836 by Beach et al. entitled “Methods and compositions for RNA interference”; WO 01/70949 by Graham et al. entitled “Genetic silencing”; and WO 01/75164 by Tuschl et al. entitled “RNA sequence-specific mediators of RNA interference.”

In brief, double-stranded RNA introduced into a cell (e.g., into the cytoplasm) is processed, for example by an RNAse III-like enzyme called Dicer, into shorter double-stranded fragments called small interfering RNAs (siRNAs, also called short interfering RNAs). The length and nature of the siRNAs produced is dependent on the species of the cell, although typically siRNAs are 21-25 nucleotides long (e.g., an siRNA may have a 19 base pair duplex portion with two nucleotide 3′ overhangs at each end). Similar siRNAs can be produced in vitro (e.g., by chemical synthesis or in vitro transcription) and introduced into the cell to induce RNAi. The siRNA becomes associated with an RNA-induced silencing complex (RISC). Separation of the sense and antisense strands of the siRNA, and interaction of the siRNA antisense strand with its target mRNA through complementary base-pairing interactions, optionally occurs. Finally, the mRNA is cleaved and degraded.

Expression of a target gene in a cell (e.g., a gene from Appendix 1) can thus be specifically inhibited by introducing an appropriately chosen double-stranded RNA into the cell. Guidelines for design of suitable interfering RNAs are known to those of skill in the art. For example, interfering RNAs are typically designed against exon sequences, rather than introns or untranslated regions. Characteristics of high efficiency interfering RNAs may vary by cell type. For example, although siRNAs may require 3′ overhangs and 5′ phosphates for most efficient induction of RNAi in Drosophila cells, in mammalian cells blunt ended siRNAs and/or RNAs lacking 5′ phosphates can induce RNAi as effectively as siRNAs with 3′ overhangs and/or 5′ phosphates (see, e.g., Czauderna et al. (2003) “Structural variations and stabilizing modifications of synthetic siRNAs in mammalian cells” Nucl Acids Res 31:2705-2716). As another example, since double-stranded RNAs greater than 30-80 base pairs long activate the antiviral interferon response in mammalian cells and result in non-specific silencing, interfering RNAs for use in mammalian cells are typically less than 30 base pairs (for example, Caplen et al. (2001) “Specific inhibition of gene expression by small double-stranded RNAs in invertebrate and vertebrate systems” Proc. Natl. Acad. Sci. USA 98:9742-9747, Elbashir et al. (2001) “Duplexes of 21-nucleotide RNAs mediate RNA interference in cultured mammalian cells” Nature 411:494-498 and Elbashir et al. (2002) “Analysis of gene function in somatic mammalian cells using small interfering RNAs” Methods 26:199-213 describe the use of 21 nucleotide siRNAs to specifically inhibit gene expression in mammalian cell lines, and Kim et al. (2005) “Synthetic dsRNA Dicer substrates enhance RNAi potency and efficacy” Nature Biotechnology 23:222-226 describes use of 25-30 nucleotide duplexes). The sense and antisense strands of a siRNA are typically, but not necessarily, completely complementary to each other over the double-stranded region of the siRNA (excluding any overhangs). The antisense strand is typically completely complementary to the target mRNA over the same region, although some nucleotide substitutions can be tolerated (e.g., a one or two nucleotide mismatch between the antisense strand and the mRNA can still result in RNAi, although at reduced efficiency). The ends of the double-stranded region are typically more tolerant to substitution than the middle; for example, as little as 15 bp (base pairs) of complementarity between the antisense strand and the target mRNA in the context of a 21 mer with a 19 bp double-stranded region has been shown to result in a functional siRNA (see, e.g., Czauderna et al. (2003) “Structural variations and stabilizing modifications of synthetic siRNAs in mammalian cells” Nucl Acids Res 31:2705-2716). Any overhangs can but need not be complementary to the target mRNA; for example, TT (two 2′-deoxythymidines) overhangs are frequently used to reduce synthesis costs.

Although double-stranded RNAs (e.g., double-stranded siRNAs) were initially thought to be required to initiate RNAi, several recent reports indicate that the antisense strand of such siRNAs is sufficient to initiate RNAi. Single-stranded antisense siRNAs can initiate RNAi through the same pathway as double-stranded siRNAs (as evidenced, for example, by the appearance of specific mRNA endonucleolytic cleavage fragments). As for double-stranded interfering RNAs, characteristics of high-efficiency single-stranded siRNAs may vary by cell type (e.g., a 5′ phosphate may be required on the antisense strand for efficient induction of RNAi in some cell types, while a free 5′ hydroxyl is sufficient in other cell types capable of phosphorylating the hydroxyl). See, e.g., Martinez et al. (2002) “Single-stranded antisense siRNAs guide target RNA cleavage in RNAi” Cell 110:563-574; Amarzguioui et al. (2003) “Tolerance for mutations and chemical modifications in a siRNA” Nucl. Acids Res. 31:589-595; Holen et al. (2003) “Similar behavior of single-strand and double-strand siRNAs suggests that they act through a common RNAi pathway” Nucl. Acids Res. 31:2401-2407; and Schwarz et al. (2002) Mol. Cell 10:537-548.

Due to currently unexplained differences in efficiency between siRNAs corresponding to different regions of a given target mRNA, several siRNAs are typically designed and tested against the target mRNA to determine which siRNA is most effective. Interfering RNAs can also be produced as small hairpin RNAs (shRNAs, also called short hairpin RNAs), which are processed in the cell into siRNA-like molecules that initiate RNAi (see, e.g., Siolas et al. (2005) “Synthetic shRNAs as potent RNAi triggers” Nature Biotechnology 23:227-231).

The presence of RNA, particularly double-stranded RNA, in a cell can result in inhibition of expression of a gene comprising a sequence identical or nearly identical to that of the RNA through mechanisms other than RNAi. For example, double-stranded RNAs that are partially complementary to a target mRNA can repress translation of the mRNA without affecting its stability. As another example, double-stranded RNAs can induce histone methylation and heterochromatin formation, leading to transcriptional silencing of a gene comprising a sequence identical or nearly identical to that of the RNA (see, e.g., Schramke and Allshire (2003) “Hairpin RNAs and retrotransposon LTRs effect RNAi and chromatin-based gene silencing” Science 301:1069-1074; Kawasaki and Taira (2004) “Induction of DNA methylation and gene silencing by short interfering RNAs in human cells” Nature 431:211-217; and Morris et al. (2004) “Small interfering RNA-induced transcriptional gene silencing in human cells” Science 305:1289-1292).

Short RNAs called microRNAs (miRNAs) have been identified in a variety of species. Typically, these endogenous RNAs are each transcribed as a long RNA and then processed to a pre-miRNA of approximately 60-75 nucleotides that forms an imperfect hairpin (stem-loop) structure. The pre-miRNA is typically then cleaved, e.g., by Dicer, to form the mature miRNA. Mature miRNAs are typically approximately 21-25 nucleotides in length, but can vary, e.g., from about 14 to about 25 or more nucleotides. Some, though not all, miRNAs have been shown to inhibit translation of mRNAs bearing partially complementary sequences. Such miRNAs contain one or more internal mismatches to the corresponding mRNA that are predicted to result in a bulge in the center of the duplex formed by the binding of the miRNA antisense strand to the mRNA. The miRNA typically forms approximately 14-17 Watson-Crick base pairs with the mRNA; additional wobble base pairs can also be formed. In addition, short synthetic double-stranded RNAs (e.g., similar to siRNAs) containing central mismatches to the corresponding mRNA have been shown to repress translation (but not initiate degradation) of the mRNA. See, for example, Zeng et al. (2003) “MicroRNAs and small interfering RNAs can inhibit mRNA expression by similar mechanisms” Proc. Natl. Acad. Sci. USA 100:9779-9784; Doench et al. (2003) “siRNAs can function as miRNAs” Genes & Dev. 17:438-442; Bartel and Bartel (2003) “MicroRNAs: At the root of plant development?” Plant Physiology 132:709-717; Schwarz and Zamore (2002) “Why do miRNAs live in the miRNP?” Genes & Dev. 16:1025-1031; Tang et al. (2003) “A biochemical framework for RNA silencing in plants” Genes & Dev. 17:49-63; Meister et al. (2004) “Sequence-specific inhibition of microRNA- and siRNA-induced RNA silencing” RNA 10:544-550; Nelson et al. (2003) “The microRNA world: Small is mighty” Trends Biochem. Sci. 28:534-540; Scacheri et al. (2004) “Short interfering RNAs can induce unexpected and divergent changes in the levels of untargeted proteins in mammalian cells” Proc. Natl. Acad. Sci. USA 101:1892-1897; Sempere et al. (2004) “Expression profiling of mammalian microRNAs uncovers a subset of brain-expressed microRNAs with possible roles in murine and human neuronal differentiation” Genome Biology 5:R13; Dykxhoorn et al. (2003) “Killing the messenger: Short RNAs that silence gene expression” Nature Reviews Molec. and Cell Biol. 4:457-467; McManus (2003) “MicroRNAs and cancer” Semin Cancer Biol. 13:253-288; and Stark et al. (2003) “Identification of Drosophila microRNA targets” PLoS Biol. 1:E60.

The cellular machinery involved in translational repression of mRNAs by partially complementary RNAs (e.g., certain miRNAs) appears to partially overlap that involved in RNAi, although, as noted, translation of the mRNAs, not their stability, is affected and the mRNAs are typically not degraded.

The location and/or size of the bulge(s) formed when the antisense strand of the RNA binds the mRNA can affect the ability of the RNA to repress translation of the mRNA. Similarly, location and/or size of any bulges within the RNA itself can also affect efficiency of translational repression. See, e.g., the references above. Typically, translational repression is most effective when the antisense strand of the RNA is complementary to the 3′ untranslated region (3′ UTR) of the mRNA. Multiple repeats, e.g., tandem repeats, of the sequence complementary to the antisense strand of the RNA can also provide more effective translational repression; for example, some mRNAs that are translationally repressed by endogenous miRNAs contain 7-8 repeats of the miRNA binding sequence at their 3′ UTRs. It is worth noting that translational repression appears to be more dependent on concentration of the RNA than RNA interference does; translational repression is thought to involve binding of a single mRNA by each repressing RNA, while RNAi is thought to involve cleavage of multiple copies of the mRNA by a single siRNA-RISC complex.

Guidance for design of a suitable RNA to repress translation of a given target mRNA can be found in the literature (e.g., the references above and Doench and Sharp (2004) “Specificity of microRNA target selection in translational repression” Genes & Dev. 18:504-511; Rehmsmeier et al. (2004) “Fast and effective prediction of microRNA/target duplexes” RNA 10:1507-1517; Robins et al. (2005) “Incorporating structure to predict microRNA targets” Proc Natl Acad Sci 102:4006-4009; and Mattick and Makunin (2005) “Small regulatory RNAs in mammals” Hum. Mol. Genet. 14:R121-R132, among many others) and herein. However, due to differences in efficiency of translational repression between RNAs of different structure (e.g., bulge size, sequence, and/or location) and RNAs corresponding to different regions of the target mRNA, several RNAs are optionally designed and tested against the target mRNA to determine which is most effective at repressing translation of the target mRNA.

### Correlating Markers to Phenotypes

One aspect of the invention is a description of correlations between polymorphisms within or linked to the genes of Appendix 1 and the various disorders and phenotypes herein (e.g., differential functional brain images, linked to neuropsychiatric disorders such as schizophrenia). An understanding of these correlations can further be used in the present invention to correlate information regarding a set of polymorphisms that an individual or sample is determined to possess and a phenotype that they are likely to display. Further, higher order correlations that account for combinations of alleles in one or more different genes in the appendix (or otherwise linked to these disorders) can also be assessed for correlations to phenotype.

These correlations can be performed by any method that can identify a relationship between an allele and a phenotype, or a combination of alleles and a combination of phenotypes. For example, alleles in one or more of the genes or loci in Appendix 1 can be correlated with one or more disorder/phenotype. Most typically, these methods involve referencing a look up table that comprises correlations between alleles of the polymorphism and the phenotype. The table can include data for multiple allele-phenotype relationships and can take account of additive or other higher order effects of multiple allele-phenotype relationships, e.g., through the use of statistical tools such as principle component analysis, heuristic algorithms, etc.

Correlation of a marker to a phenotype optionally includes performing one or more statistical tests for correlation. Many statistical tests are known, and most are computer-implemented for ease of analysis. A variety of statistical methods of determining associations/correlations between phenotypic traits and biological markers are known and can be applied to the present invention. For an introduction to the topic, see, Hartl (1981) A Primer of Population Genetics Washington University, Saint Louis Sinauer Associates, Inc. Sunderland, Mass. ISBN: 0-087893-271-2. A variety of appropriate statistical models are described in Lynch and Walsh (1998) Genetics and Analysis of Quantitative Traits, Sinauer Associates, Inc. Sunderland Mass. ISBN 0-87893-481-2. These models can, for example, provide for correlations between genotypic and phenotypic values, characterize the influence of a locus on a phenotype, sort out the relationship between environment and genotype, determine dominance or penetrance of genes, determine maternal and other epigenetic effects, determine principle components in an analysis (via principle component analysis, or “PCA”), and the like. The references cited in these texts provides considerable further detail on statistical models for correlating markers and phenotype.

In addition to standard statistical methods for determining correlation, other methods that determine correlations by pattern recognition and training, such as the use of genetic algorithms, can be used to determine correlations between markers and phenotypes. This is particularly useful when identifying higher order correlations between multiple alleles and multiple phenotypes, e.g., once basic correlations between alleles and phenotypes have been made. To illustrate, neural network approaches can be coupled to genetic algorithm-type programming for heuristic development of a structure-function data space model that determines correlations between genetic information and phenotypic outcomes. For example, NNUGA (Neural Network Using Genetic Algorithms) is an available program (e.g., on the world wide web at cs.bgu.ac.il/˜omri/NNUGA which couples neural networks and genetic algorithms. An introduction to neural networks can be found, e.g., in Kevin Gurney, An Introduction to Neural Networks, UCL Press (1999) and on the world wide web at shef.ac.uk/psychology/gurney/notes/index.html. Additional useful neural network references include those noted above in regard to genetic algorithms and, e.g., Bishop, Neural Networks for Pattern Recognition, Oxford University Press (1995), and Ripley et al., Pattern Recognition and Neural Networks, Cambridge University Press (1995).

Additional references that are useful in understanding data analysis applications for using and establishing correlations, principle components of an analysis, neural network modeling and the like, include, e.g., Hinchliffe, Modeling Molecular Structures, John Wiley and Sons (1996), Gibas and Jambeck, Bioinformatics Computer Skills, O'Reilly (2001), Pevzner, Computational Molecular Biology and Algorithmic Approach, The MIT Press (2000), Durbin et al., Biological Sequence Analysis: Probabilistic Models of Proteins and Nucleic Acids, Cambridge University Press (1998), and Rashidi and Buehler, Bioinformatic Basics: Applications in Biological Science and Medicine, CRC Press LLC (2000).

In any case, essentially any statistical test can be applied in a computer implemented model, by standard programming methods, or using any of a variety of “off the shelf” software packages that perform such statistical analyses, including, for example, those noted above and those that are commercially available, e.g., from Partek Incorporated (St. Peters, Mo.; www.partek.com), e.g., that provide software for pattern recognition (e.g., which provide Partek Pro 2000 Pattern Recognition Software) which can be applied to genetic algorithms for multivariate data analysis, interactive visualization, variable selection, neural network & statistical modeling, etc. Relationships can be analyzed, e.g., by Principal Components Analysis (PCA) mapped mapped scatterplots and biplots, Multi-Dimensional Scaling (MDS) Multi-Dimensional Scaling (MDS) mapped scatterplots, star plots, etc. Available software for performing correlation analysis includes SAS, R and MathLab.

In any case, the marker(s), whether polymorphisms or expression patterns, can be used for any of a variety of genetic analyses. For example, once markers have been identified, as in the present case, they can be used in a number of different assays for association studies. For example, probes can be designed for microarrays that interrogate these markers. Other exemplary assays include, e.g., the Taqman assays and molecular beacon assays described supra, as well as conventional PCR and/or sequencing techniques.

In some embodiments, the marker data is used to perform association studies to show correlations between markers and phenotypes. This can be accomplished by determining marker characteristics in individuals with the phenotype of interest (i.e., individuals or populations displaying the phenotype of interest) and comparing the allele frequency or other characteristics (expression levels, etc.) of the markers in these individuals to the allele frequency or other characteristics in a control group of individuals. Such marker determinations can be conducted on a genome-wide basis, or can be focused on specific regions of the genome (e.g., haplotype blocks of interest). In one embodiment, markers that are linked to the genes of Appendix 1 are assessed for correlation to one or more specific phenotypes.

In one aspect, the invention includes the use of a general linear model (GLM) that combines differential brain imaging phenotypes, disease diagnosis, and genetic data in a single model:

Imaging Phenotype=Genotype Effect+Diagnosis Effect+Genotype-Diagnosis Interaction Effect.

This model was used to identify the correlations between the genes in Appendix 1 and functional differential brain images (detected by fMRI), that, in turn are linked to neuropsychiatric disorders such as schizophrenia (see, the Examples section below). This method can be used to identify additional correlations, e.g., by determining differential brain image differences for other neuropsychiatric disorders such as other psychotic disorders, bipolar disorder, mood disorders such as major or clinical depression, anxiety disorders such as generalized anxiety disorder, somatoform disorders (Briquet's disorder), factitious disorders such as Munchausen syndrome, dissociative disorders such as dissociative identity disorder, sexual disorders such as dyspareunia and gender identity disorder, eating disorders such as anorexia nervosa, sleep disorders such as insomnia and narcolepsy, impulse control disorders such as kleptomania, adjustment disorders, personality disorders such as narcissistic personality disorder, tardive dyskinesia, tourettes, autism, dyslexia, dyspraxia, hyperactivity and many others.

Once these brain image differences are identified for a given disorder they can be assigned a quantitative value. That is, differences in brain activation are measured in the patient population, as compared to a control population for different functional test conditions (e.g., high and low load memory tests). A difference between the brain image under the first and second condition is determined and a summary statistic is assigned to quantify the difference in functional activation.

The GLM is then used to correlate the genotype and the phenotype. Further details regarding this general method are found in the Examples below.

In addition to the other embodiments of the methods of the present invention disclosed herein, the methods additionally allow for the “dissection” of a phenotype. That is, a particular phenotype can result from two or more different genetic causes. For example, a neuropsychiatric disorder may be the result of a “defect” (or simply a particular allele—“defect” with respect to a susceptibility phenotype is context dependent, e.g., whether the phenotype is desirable or undesirable in the individual in a given environment) in a gene of Appendix 1, while the same basic phenotype in a different individual may be the result of multiple “defects” in one or more of these genes. Thus, scanning a plurality of markers (e.g., as in genome or haplotype block scanning) allows for the dissection of varying genetic bases for similar (or graduated) phenotypes.

As described in the previous paragraph, one method of conducting association studies is to compare the allele frequency (or expression level) of markers in individuals with a phenotype of interest (“case group,” e.g., characterized patients that are diagnosed as suffering from a neuropsychiatric disorder such as schizophrenia) to the allele frequency in a control group of individuals (e.g., cognitively and psychiatrically healthy individuals). In one method, informative SNPs are used to make the SNP haplotype pattern comparison (an “informative SNP” is genetic SNP marker such as a SNP or subset (more than one) of SNPs in a genome or haplotype block that tends to distinguish one SNP or genome or haplotype pattern from other SNPs, genomes or haplotype patterns).

Thus, in an embodiment of one method of determining genetic associations, the allele frequency of informative SNPs is determined for genomes of a control population that do not display the disorder (or brain image phenotype). The allele frequency of informative SNPs is also determined for genomes of a population that do display the phenotype. The informative SNP allele frequencies are compared. Allele frequency comparisons can be made, for example, by determining the allele frequency (number of instances of a particular allele in a population divided by the total number of alleles) at each informative SNP location in each population and comparing these allele frequencies. The informative SNPs displaying a difference between the allele frequency of occurrence in the control versus case populations/groups are selected for analysis. Once informative SNPs are selected, the SNP haplotype block(s) that contain the informative SNPs are identified, which in turn identifies a genomic region of interest that is correlated with the phenotype. The genomic regions can be analyzed by genetic or any biological methods known in the art e.g., for use as drug discovery targets or as diagnostic markers.

### Systems for Identifying a Phenotype or Neuropsychiatric Disorder

Systems for performing the above correlations are also a feature of the invention. Typically, the system will include system instructions that correlate the presence or absence of an allele (whether detected directly or, e.g., through expression levels) with a predicted differential brain image phenotype or neuropsychiatric disorder. The system instructions can compare detected information as to allele sequence or expression level with a database that includes correlations between the alleles and the relevant phenotypes/disorders. This database can be multidimensional, thereby including higher-order relationships between combinations of alleles and the relevant phenotypes/disorders. These relationships can be stored in any number of look-up tables, e.g., taking the form of spreadsheets (e.g., Excel™ spreadsheets) or databases such as an Access™, SQL™, Oracle™, Paradox™, or similar database. The system includes provisions for inputting sample-specific information regarding allele detection information, e.g., through an automated or user interface and for comparing that information to the look up tables.

Optionally, the system instructions can also include software that accepts diagnostic information associated with any detected allele information, e.g., a diagnosis that a subject with the relevant allele has a particular brain image phenotype or disorder. This software can be heuristic in nature, using such inputted associations to improve the accuracy of the look up tables and/or interpretation of the look up tables by the system. A variety of such approaches, including GLM, neural networks, Markov modeling, and other statistical analysis are described above.

The invention provides data acquisition modules for detecting one or more detectable genetic marker(s) (e.g., one or more array comprising one or more biomolecular probes, detectors, fluid handlers, or the like). The biomolecular probes of such a data acquisition module can include any that are appropriate for detecting the biological marker, e.g., oligonucleotide probes, proteins, aptamers, antibodies, etc. These can include sample handlers (e.g., fluid handlers), robotics, microfluidic systems, nucleic acid or protein purification modules, arrays (e.g., nucleic acid arrays), detectors, thermocyclers or combinations thereof, e.g., for acquiring samples, diluting or aliquoting samples, purifying marker materials (e.g., nucleic acids or proteins), amplifying marker nucleic acids, detecting amplified marker nucleic acids, and the like.

For example, automated devices that can be incorporated into the systems herein have been used to assess a variety of biological phenomena, including, e.g., expression levels of genes in response to selected stimuli (Service (1998) “Microchips Arrays Put DNA on the Spot” Science 282:396-399), high throughput DNA genotyping (Zhang et al. (1999) “Automated and Integrated System for High-Throughput DNA Genotyping Directly from Blood” Anal. Chem. 71:1138-1145) and many others. Similarly, integrated systems for performing mixing experiments, DNA amplification, DNA sequencing and the like are also available. See, e.g., Service (1998) “Coming Soon: the Pocket DNA Sequencer” Science 282: 399-401. A variety of automated system components are available, e.g., from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). Similarly, commercially available microfluidic systems that can be used as system components in the present invention include those from Agilent technologies and the Caliper Technologies. Furthermore, the patent and technical literature includes numerous examples of microfluidic systems, including those that can interface directly with microwell plates for automated fluid handling.

Any of a variety of liquid handling and/or array configurations can be used in the systems herein. One common format for use in the systems herein is a microtiter plate, in which the array or liquid handler includes a microtiter tray. Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use. Samples can be processed in such trays, with all of the processing steps being performed in the trays. Samples can also be processed in microfluidic apparatus, or combinations of microtiter and microfluidic apparatus.

In addition to liquid phase arrays, components can be stored in or analyzed on solid phase arrays. These arrays fix materials in a spatially accessible pattern (e.g., a grid of rows and columns) onto a solid substrate such as a membrane (e.g., nylon or nitrocellulose), a polymer or ceramic surface, a glass or modified silica surface, a metal surface, or the like. Components can be accessed, e.g., by hybridization, by local rehydration (e.g., using a pipette or other fluid handling element) and fluidic transfer, or by scraping the array or cutting out sites of interest on the array.

The system can also include detection apparatus that is used to detect allele information, using any of the approached noted herein. For example, a detector configured to detect real-time PCR products (e.g., a light detector, such as a fluorescence detector) or an array reader can be incorporated into the system. For example, the detector can be configured to detect a light emission from a hybridization or amplification reaction comprising an allele of interest, wherein the light emission is indicative of the presence or absence of the allele. Optionally, an operable linkage between the detector and a computer that comprises the system instructions noted above is provided, allowing for automatic input of detected allele-specific information to the computer, which can, e.g., store the database information and/or execute the system instructions to compare the detected allele specific information to the look up table.

Probes that are used to generate information detected by the detector can also be incorporated within the system, along with any other hardware or software for using the probes to detect the amplicon. These can include thermocycler elements (e.g., for performing PCR or LCR amplification of the allele to be detected by the probes), arrays upon which the probes are arrayed and/or hybridized, or the like. The fluid handling elements noted above for processing samples, can be used for moving sample materials (e.g., template nucleic acids and/or proteins to be detected) primers, probes, amplicons, or the like into contact with one another. For example, the system can include a set of marker probes or primers configured to detect at least one allele of one or more genes or linked loci associated with a phenotype or disorder as noted herein, where the gene is as listed in Appendix 1. The detector module is configured to detect one or more signal outputs from the set of marker probes or primers, or an amplicon produced from the set of marker probes or primers, thereby identifying the presence or absence of the allele.

The sample to be analyzed is optionally part of the system, or can be considered separate from it. The sample optionally includes e.g., genomic DNA, amplified genomic DNA, cDNA, amplified cDNA, RNA, amplified RNA, proteins, etc., as noted herein. In one aspect, the sample is derived from a mammal such as a human or veterinary patient.

Optionally, system components for interfacing with a user are provided. For example, the systems can include a user viewable display for viewing an output of computer-implemented system instructions, user input devices (e.g., keyboards or pointing devices such as a mouse) for inputting user commands and activating the system, etc. Typically, the system of interest includes a computer, wherein the various computer-implemented system instructions are embodied in computer software, e.g., stored on computer readable media.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Sequel™, Oracle™, Paradox™) can be adapted to the present invention by inputting a character string corresponding to an allele herein, or an association between an allele and a phenotype. For example, the systems can include software having the appropriate character string information, e.g., used in conjunction with a user interface (e.g., a GUI in a standard operating system such as a Windows, Macintosh or LINUX system) to manipulate strings of characters. Specialized sequence alignment programs such as BLAST can also be incorporated into the systems of the invention for alignment of nucleic acids or proteins (or corresponding character strings) e.g., for identifying and relating alleles.

As noted, systems can include a computer with an appropriate database and an allele sequence or correlation of the invention. Software for aligning sequences, as well as data sets entered into the software system comprising any of the sequences herein can be a feature of the invention. The computer can be, e.g., a PC (Intel x86 or Pentium chip-compatible DOS™, OS2™ WINDOWS™ WINDOWS NT™, WINDOWS95™, WINDOWS98™, WINDOWS2000, WINDOWSME, or LINUX based machine, a MACINTOSH™, Power PC, or a UNIX based (e.g., SUN™ work station or LINUX based machine) or other commercially common computer which is known to one of skill. Software for entering and aligning or otherwise manipulating sequences is available, e.g., BLASTP and BLASTN, or can easily be constructed by one of skill using a standard programming language such as Visualbasic, Fortran, Basic, Java, or the like.

### Methods of Identifying Modulators

In addition to providing various diagnostic and prognostic markers for identifying neuropsychiatric disorders, etc., the invention also provides methods of identifying modulators of these disorders. In the methods, a potential modulator is contacted to a relevant protein (encoded by a gene of Appendix 1) or to a nucleic acid that encodes such a protein. An effect of the potential modulator on the gene or gene product is detected, thereby identifying whether the potential modulator modulates an underlying molecular cause of the disorder.

In addition, the methods can include, e.g., administering one or more putative modulator to an individual that displays a relevant phenotype and determining whether the putative modulator modulates the phenotype in the individual, e.g., in the context of a clinical trial or treatment. This, in turn, determines whether the putative modulator is clinically useful.

The gene or gene product that is contacted by the modulator can include any allelic form noted herein. Allelic forms, whether genes or proteins, that positively correlate to undesirable phenotypes or disorders are preferred targets for modulator screening.

Effects of interest that can be screened for include: (a.) increased or decreased expression of the gene in the presence of the modulator; (b.) a change in localization of the gene product in the presence of the modulator; (c.) a change in an activity of a RHO-GTPase encoded by an ARHGAP18 gene; and, (d.) a change in RAS or EGFR-mediated cell proliferation, migration or differentiation.

The precise format of the modulator screen will, of course, vary, depending on the effect(s) being detected and the equipment available. Northern analysis, quantitative RT-PCR and/or array-based detection formats can be used to distinguish expression levels of genes noted above. Protein expression levels can also be detected using available methods, such as western blotting, ELISA analysis, antibody hybridization, BIAcore, or the like. Any of these methods can be used to distinguish changes in expression levels of a gene or protein of interest, e.g., that results from activity of a potential modulator.

Accordingly, one may screen for potential modulators of genes or gene products of Appendix 1 for activity or expression. For example, potential modulators (small molecules, organic molecules, inorganic molecules, proteins, hormones, transcription factors, or the like) can be contacted to a cell comprising an allele of interest and an effect on activity or expression (or both) of a gene or gene product of Appendix 1 can be detected. For example, expression of a gene or interest can be detected, e.g., via northern analysis or quantitative (optionally real time) RT-PCR, before and after application of potential expression modulators. Similarly, promoter regions of the various genes (e.g., generally sequences in the region of the start site of transcription, e.g., within 5 KB of the start site, e.g., 1 KB, or less e.g., within 500 BP or 250 BP or 100 BP of the start site) can be coupled to reporter constructs (CAT, beta-galactosidase, luciferase or any other available reporter) and can be similarly be tested for expression activity modulation by the potential modulator. In either case, the assays can be performed in a high-throughput fashion, e.g., using automated fluid handling and/or detection systems, in serial or parallel fashion. Similarly, activity modulators can be tested by contacting a potential modulator to an appropriate cell using any of the activity detection methods herein, regardless of whether the activity that is detected is the result of activity modulation, expression modulation or both. These assays can be in vitro, cell-based, or can be screens for modulator activity performed on laboratory animals such as knock-out transgenic mice comprising a gene of interest.

Biosensors for detecting modulator activity detection are also a feature of the invention. These include devices or systems that comprise a gene or gene product of Appendix 1 coupled to a readout that measures or displays one or more activity of the protein or gene. Thus, any of the above described assay components can be configured as a biosensor by operably coupling the appropriate assay components to a readout. The readout can be optical (e.g., to detect cell markers or cell survival) electrical (e.g., coupled to a FET, a BIAcore, or any of a variety of others), spectrographic, or the like, and can optionally include a user-viewable display (e.g., a CRT or optical viewing station). The biosensor can be coupled to robotics or other automation, e.g., microfluidic systems, that direct contact of the putative modulators to the proteins of the invention, e.g., for automated high-throughput analysis of putative modulator activity. A large variety of automated systems that can be adapted to use with the biosensors of the invention are commercially available. For example, automated systems have been made to assess a variety of biological phenomena, including, e.g., expression levels of genes in response to selected stimuli (Service (1998) “Microchips Arrays Put DNA on the Spot” Science 282:396-399). Laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays and control a variety of environmental conditions such as temperature, exposure to light or air, and the like. Many such automated systems are commercially available and are described herein, including those described above. These include various Zymate systems, ORCA® robots, microfluidic devices, etc. For example, the LabMicrofluidic Device® high throughput screening system (HTS) by Caliper Technologies, Mountain View, Calif. can be adapted for use in the present invention to screen for modulator activity.

In general, methods and sensors for detecting protein expression level and activity are available, including those taught in the various references above, including R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and Satinder Ahuja ed., Handbook of Bioseparations, Academic Press (2000). “Proteomic” detection methods, which detect many proteins simultaneously have been described and are also noted above, including various multidimensional electrophoresis methods (e.g., 2-d gel electrophoresis), mass spectrometry based methods (e.g., SELDI, MALDI, electrospray, etc.), or surface plasmon resonance methods. These can also be used to track protein activity and/or expression level.

Similarly, nucleic acid expression levels (e.g., mRNA) can be detected using any available method, including northern analysis, quantitative RT-PCR, or the like. References sufficient to guide one of skill through these methods are readily available, including Ausubel, Sambrook and Berger.

Whole animal assays can also be used to assess the effects of modulators on cells or whole animals (e.g., transgenic knock-out mice), e.g., by monitoring an effect on a cell-based phenomenon, a change in displayed animal phenotype, or the like.

Potential modulator libraries to be screened for effects genes or gene products are available. These libraries can be random, or can be targeted.

Targeted libraries include those designed using any form of a rational design technique that selects scaffolds or building blocks to generate combinatorial libraries. These techniques include a number of methods for the design and combinatorial synthesis of target-focused libraries, including morphing with bioisosteric transformations, analysis of target-specific privileged structures, and the like. In general, where information regarding structure of genes of Appendix 1 is available, likely binding partners can be designed, e.g., using flexible docking approaches, or the like. Similarly, random libraries exist for a variety of basic chemical scaffolds. In either case, many thousands of scaffolds and building blocks for chemical libraries are available, including those with polypeptide, nucleic acid, carbohydrate, and other backbones.

Kits for treatment of a disorder can include a modulator identified as noted above and instructions for administering the compound to a patient to the disorder.

### Cell Rescue and Therapeutic Administration

In one aspect, the invention includes rescue of a cell that is defective in function of one or more endogenous genes or polypeptides for a gene of appendix 1, or administration of an inhibitor such as an RNAi moiety that inhibits expression. This can be accomplished simply by introducing a new copy of the gene (or a heterologous nucleic acid that expresses the relevant protein), i.e., a gene having an allele that is desired, into the cell, or by introducing an expression construct that comprises the inhibitor into the cell. Other approaches, such as homologous recombination to repair the defective gene (e.g., via chimeraplasty) can also be performed. In any event, rescue of function can be measured, e.g., in any of the assays noted herein. Indeed, this method can be used as a general method of screening cells in vitro for expression or activity of any gene or gene product of Appendix 1.

Accordingly, in vitro rescue of function is useful in this context for the myriad in vitro screening methods noted above. The cells that are rescued can include cells in culture, (including primary or secondary cell culture from patients, as well as cultures of well-established cells). Where the cells are isolated from a patient, this has additional diagnostic utility in establishing which Appendix 1 sequence is defective in a patient that presents with a relevant phenotype.

In another aspect, the cell rescue occurs in a patient, e.g., a human or veterinary patient, e.g., to remedy a disorder or disorder predisposition. Thus, one aspect of the invention is gene therapy to remedy disorders, in human or veterinary applications. In these applications, the nucleic acids of the invention (including genes or inhibitors) are optionally cloned into appropriate gene therapy vectors (and/or are simply delivered as naked or liposome-conjugated nucleic acids), which are then delivered, optionally in combination with appropriate carriers or delivery agents. Proteins can also be delivered directly, but delivery of the nucleic acid is typically preferred in applications where stable expression is desired. Similarly, modulators of any metabolic defect that are identified by the methods herein can be used therapeutically.

Compositions for administration, e.g., comprise a therapeutically effective amount of the modulator, gene therapy vector or other relevant nucleic acid, and a pharmaceutically acceptable carrier or excipient. Such a carrier or excipient includes, but is not limited to, saline, buffered saline, dextrose, water, glycerol, ethanol, and/or combinations thereof. The formulation is made to suit the mode of administration. In general, methods of administering gene therapy vectors for topical use are well known in the art and can be applied to administration of the nucleic acids of the invention.

Therapeutic compositions comprising one or more modulator or gene therapy nucleic acid of the invention are optionally tested in one or more appropriate in vitro and/or in vivo animal model of disease, to confirm efficacy, tissue metabolism, and to estimate dosages, according to methods well known in the art. In particular, dosages can initially be determined by activity, stability or other suitable measures of the formulation.

Administration is by any of the routes normally used for introducing a molecule into ultimate contact with cells. Modulators and/or nucleic acids that encode genes of Appendix 1, or inhibitors thereof, can be administered in any suitable manner, optionally with one or more pharmaceutically acceptable carriers. Suitable methods of administering such nucleic acids in the context of the present invention to a patient are available, and, although more than one route can be used to administer a particular composition, a particular route can often provide a more immediate and more effective action or reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention. Compositions can be administered by a number of routes including, but not limited to: oral, intravenous, intraperitoneal, intramuscular, transdermal, subcutaneous, topical, sublingual, or rectal administration. Compositions can be administered via liposomes (e.g., topically), or via topical delivery of naked DNA or viral vectors. Such administration routes and appropriate formulations are generally known to those of skill in the art.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multi-dose sealed containers, such as ampules and vials.

The dose administered to a patient, in the context of the present invention, is sufficient to effect a beneficial therapeutic response in the patient over time. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, the physician evaluates local expression, or circulating plasma levels, formulation toxicities, progression of the relevant disease, and/or where relevant, the production of antibodies to proteins encoded by the polynucleotides. The dose administered, e.g., to a 70 kilogram patient are typically in the range equivalent to dosages of currently-used therapeutic proteins, adjusted for the altered activity or serum half-life of the relevant composition. The vectors of this invention can supplement treatment conditions by any known conventional therapy.

For administration, formulations of the present invention are administered at a rate determined by the LD-50 of the relevant formulation, and/or observation of any side-effects of the vectors of the invention at various concentrations, e.g., as applied to the mass or topical delivery area and overall health of the patient. Administration can be accomplished via single or divided doses.

If a patient undergoing treatment develops fevers, chills, or muscle aches, he/she receives the appropriate dose of aspirin, ibuprofen, acetaminophen or other pain/fever controlling drug. Patients who experience reactions to the compositions, such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Treatment is slowed or discontinued depending upon the severity of the reaction.

### EXAMPLES

The following examples are illustrative and not limiting. One of skill will recognize a variety of parameters that can be modified to achieve essentially similar results.

### Example

### Proof of Concept and Discovery of ARHGAP 18 Association

Overview

Genome wide scans (GWS) offer the potential to discover unknown genes associated with neuropsychiatric illness, thereby avoiding the tautological limitation of candidate gene approaches. Obstacles to such gene wide association studies are the high likelihood of finding false positives and the very large number of subjects needed to address statistical uncertainty. In this example, we provide a strategy that combines brain imaging and GWS in a general linear model (GLM) analysis to produce imaging-gene-phenotypes (IGP) or the prediction of brain activation patterns by variations in single nucleotide polymorphisms, or SNPs.

A proof of concept example is described in which SNPs related to the gene, ARHGAP18, are associated with prefrontal activation in schizophrenia. Five of 15 SNPs that map to ARHGAP18 exceeded the permutational determined threshold of p<10⁻⁵ for activation of BA 46. The IGP associated with activation of BA 46 was also associated with activation in the other prefrontal circuitry including the BA 46, DLPFC BA 9, DPFC and to a lesser extent the neuroanatomically connected BA 6 (dorsal premotor), BA 8 (posterior dorsal prefrontal cortex) and BA 7 (superior parietal lobule), but not the caudate or thalamus. The RHO-GTPase family of genes are linked to RAS and EGFR-mediated neuronal proliferation, migration, and differentiation; the location of this gene is contained within 6q22-24, a region previously linked to schizophrenia, but this gene has not been previously identified in the literature. We provide a GWS data reduction strategy through a series of GLM analyses that identify the relationship between genetic variation and brain activation. This hierarchical stepwise approach reduces false positives, requires feasible sample sizes, and links genes and brain activation, but requires a confirmatory sample.

Introduction

Genome-wide scans offer enormous promise in identifying genetic variation involved with illness and its response to treatment. Paradoxically, as the number of variations increase, making it more likely to find the important variations, so does the likelihood of spurious findings or false positives. Solutions to this problem have been to increase the sample size to 10s of thousands or more; to increase the significance threshold astronomically; or to limit the number of single nucleotide polymorphisms considered to a priori candidates.

Each of these approaches is limited. For many illnesses, very large sample sizes are impractical. Increasing the significance level decreases the risk of false positives but brings with it the risk of false negatives. Candidate gene approaches suffer from the tautology of “only looking for what you know”, and decrease the likelihood of identifying genes with heretofore unknown functions that may be the most relevant. The point of GWS is to allow genes to be identified whose relationship with the disease phenotype has not even been hypothesized.

Our approach is to use empirically-based, brain imaging differences between the target population and healthy controls, as phenotypes to constrain the GWS analysis. Specifically, in imaging studies of neuropsychiatric patients and controls, differential activation in certain regions of interest or circuits can be identified. We limit our imaging phenotypes to these areas, and then examine the role of individual genetic variation on these phenotypes at an individual level.

This method excludes genes or polymorphisms that do not influence differences in brain area activation, or the particular imaging phenotypes chosen. However, brain imaging is a sensitive measure of brain function in neuropyschiatric illness. Thus, using an imaging phenotype has facial validity and biological relevance as it constrains the GWS analyses. On the other hand, constraints based on the sample size or significance threshold corrections have no biological relationship to the disease under study.

Nevertheless, our approach also has address issues of power and false positives. We do this through adhering to three practices: First, we require that any SNP which shows a significant relationship to the imaging phenotype not be an isolated result, but that nearby SNPs on the same gene should also show a relationship, even if it is a weaker one. Second, anatomically and/or functionally connected regions in the brain should show a similar pattern of genotype influence. Finally, these identified SNPs become candidates which must be replicated in an independent sample.

We provide an example of this method applied to a pilot study of a genome-wide scan, on a small group of schizophrenic subjects who underwent fMRI. In addition to offering a data-reduction strategy, integrating imaging and genetic measures offers clear advantages. The function of genes expressed in the brain can be revealed in neuroimaging data, and neuroimaging may identify disease phenotypes (e.g., relative functional levels of various cortical and subcortical regions) that are more closely related to susceptibility genes than are current clinical subcategorizations. Since many neuropsychiatric illnesses such as schizophrenia and bipolar disorder have clear genetic components, without considering the genetic influences the interpretation of imaging data is limited. Given the known importance of genetics in brain function, and the role of neuroimaging in revealing brain dysfunction, combining these two methods offers a new strategy and methodology for exploring genetic roles in neuropsychiatric illness.

However, there is no consensus on the most appropriate methods of such integration. To fully realize the promise of this synergy, we developed novel analytic, statistical, and visualization techniques for this new field.

Methods

The TIGC began with a well-characterized legacy dataset of 28 chronic schizophrenic subjects who had undergone cognitive assessment, clinical assessment, and functional and structural MRI, as well as blood draws for genotyping. The functional MRI tasks included a working memory task, in which subjects had to briefly remember several items; and the primary analysis was the effect of memory load on the BOLD signal.

Subjects. The sample consisted of chronic, stable patients with schizophrenia who were treated with anti-psychotic medications. Twenty-four schizophrenic patients (eight female) were recruited as part of a larger study. All subjects were medically stable. Eighteen subjects were right handed. The average age was 43 (range 27 and 60 years old). The mean duration of illness was 13.6 years (range 1 to 32 years). All were treated with stable of antipsychotic drugs, all except two with atypical antipsychotic agents. Six subjects were also on mood stabilizers, 4 on antidepressants, and 2 on antiparkinson agents.

The mean Positive and Negative Symptoms Scale (PANSS) total score was 72 (ranging from 48 to 104, with a standard deviation of 14). The negative symptom scale scores ranged from 9 to 26, with a mean and standard deviation of 19 and 4.3, respectively; the positive symptom scale scores ranged from 9 to 28, with a mean and standard deviation of 16 and 4.5.

Twenty subjects were Caucasian (3 Hispanic), and 2 African-Americans. While this is a small sample, it is typical of chronic schizophrenic patients with stable symptoms in treatment.

fMRI methods. During an fMRI scanning session using a T2*-weighted gradient echo sequence (24 cm FOV, 28 slices, 5 mm thick with no gap, interleaved, axially oriented; TR=3s, TE=40 ms, 90 deg flip angle), subjects performed three runs of a Serial Item Recognition Paradigm, a working memory task (based on Manoach et al., 1999). The task included two memory loads (2 digits and 5 digits to remember) and a control condition (left and right pointing arrows, to control for movement activations).

fMRI analyses. The fMRI data were motion-corrected, normalized to a standard space, smoothed using an 8-mm FWHM Gaussian filter, and analyzed using SPM2 (http://www.fil.ion.ucl.ac.uk/spm/). The General Linear Model (GLM) modeled the effects of the low and high memory load relative to the control condition. The contrast of interest compared the high memory load against the low memory load.

The primary region of interest (ROI) was the Left Hemisphere Brodmann Area 46, a key player in working memory studies that distinguish schizophrenics from non-schizophrenic subjects. This region is in the center of the middle frontal gyrus, corresponding largely to the dorsolateral prefrontal cortex (DLPFC).

This ROI and nine other standardized regions of interest in the cortex and subcortex were extracted using a Talairach atlas (http://www.mrc-cbu.cam.ac.uk/Imaging/Common/mnispace.shtml). A summary statistic for each region was calculated (a mean beta value for the high memory load>low memory load contrast). These summary statistics, reflecting differential imaging signals, were used as the initial imaging phenotypes.

The other areas were chosen that play a role in memory processing (Left Hemisphere BA 6 (premotor cortex), 7 (superior parietal lobule), and 8 (frontal eye fields/premotor cortex), BA24 (Left Anterior Cingulate)), as well as some that are densely anatomically connected but not necessarily involved in memory processing (Left Whole Thalamus, Caudate, and Amygdala, and Right Cerebellum). The choice of ROIs and hemisphere is based on the extensive literature implicating left hemisphere and particularly DLPFC dysfunction in schizophrenia (Fallon et al., 2003 and neg symptoms Potkin et al 200*). The right cerebellum was chosen for its known contra-lateral connectivity. We focused primarily on the BA46 results.

Imaging genetics analysis. The genetic datasets include the output of an Illumina Human-1 Genotyping Bead Chip, in an analysis performed by the Broad Institutes's Genetic Analysis Platform (http://www.broad.mit.edu/gen_analysis/genotyping/). Call rates per subject ranged from 97 to 99%, with a mean of 98.3%.

For each SNP in the 109K genome-wide scan, we performed a QTL analysis using the QTLSNP algorithm on the imaging phenotype. QTLSNP uses linear regression to compare the equality of means across genotypes while allowing for covariate adjustment. It assumes a codominant genetic model and tests an additive effect, a dominant effect, and that both effects are equal to zero (equivalent to comparing means across the three possible genotypes). Essentially, QTLSNP tests in several related ways for the influences of SNPs on imaging phenotype.

This analysis consisted of 109,000 SNPs being tested against the DLPFC imaging measure, for a total of approximately three hundred thousand statistical tests. The conservative Bonferroni correction for multiple tests requires that “significant” IGPs pass the p<10⁻⁵ level. At a level of p<10⁻⁵, by chance, we would expect three significant results.

To gauge the strength of these results, we simulated the behavior of 550,000 t-tests with this sample size, and found the smallest p value to arise by chance was p<10⁻⁵.

Results

Using the DLPFC measure as the imaging phenotype, twenty-eight genes were identified by having at least one SNP whose QTL analysis was significant at p<10⁻⁵. The evidence for a SNP playing a role in the imaging phenotype, however, is greatly strengthened by the presence of other SNPs within the same gene that show some evidence of affecting the imaging phenotype. This argument is analogous to the nearest neighbor approach for determining significant voxels in brain imaging analyses. We used as an initial rule of thumb that 25% of the remaining SNPs within the gene should be significant at least p<10−3.

A total of 13 IGPs passed the p<10⁻⁵ correction level for at least one SNP, and had 25% of the remaining SNPs within the gene significant at the p<0.001 level. All of the genes represented by these SNPs were expressed in the brain, which is not entirely surprising given that roughly half of all genes are expressed in brain.

In the DLPFC, SNP RS9372944 affected activation at p<10⁻⁷. RS9372944 is one of 11 SNPs that map the gene ARHGAP18 on chromosome 6. An additional 4 SNPs were significant with this imaging phenotype, i.e., 4 of 11 possible SNPs for ARHGAP18 at p<10⁻³.

Circuitry exploration. Given a significant IGP, it is desirable to look for the effect of the significant locus across other brain regions. This entails determining if the effects of that locus across the brain might follow the pattern of known brain circuitry or if it appears random. These SNPs were significantly associated with brain activation and corresponding implied circuitry—i.e., the S9385523 SNP alleles were clearly associated with activation in the dorsal prefrontal cortices (BA 46 DLPFC, 9 DPFC) and to a lesser extent the neuroanatomically connected BA 6 (dorsal premotor), BA 8 (posterior dorsal prefrontal cortex) and BA 7 (superior parietal lobule), but not the caudate or thalamus.

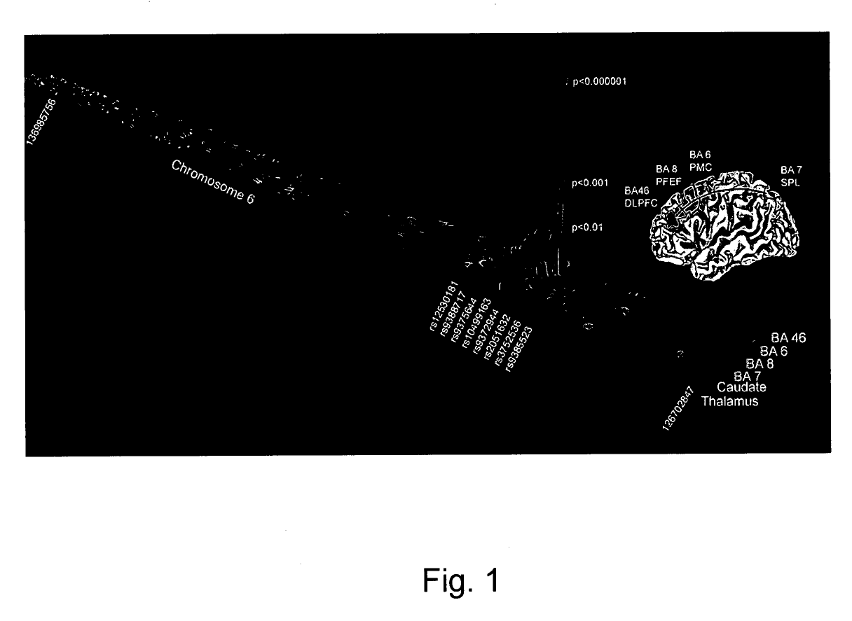

FIG. 1 shows the distribution of p values across a single portion of chromosome 6, by brain area. The pattern of peaks (low p values) is localized to one area of chromosome 6, and appears strongly in BA 46 and functionally related brain areas, but much more weakly in control areas. Additionally, the number of statistically significant SNPs in this region of 10 million bp is generally limited to this gene, rather than randomly distributed.

FIG. 1 represents p values (plotted as −log p) for all SNPs represented on the Illumina Human-1 Genotyping Bead Chip over an approximately 10 million basepair region of chromosome 6 with flanking basepair numbers indicated. Each line represents a different region of brain activation. The specific RS number for SNPs coincident with the main peaks are listed in their approximate locations. The MRI template demonstrates the implied circuitry for brain areas represented in FIGURE.

Genetic Annotation. The 2 most significant SNPs that related to BA 46 are RS9372944 (p<10⁻⁶) and RS9385523 (p<0.0025). Exploring genetic databases (e.g., dbSNP, Ensembl, SWISSPROT) revealed a lack of annotation. However, we found RS9372944 to be intronic and RS9385523 in the untranslated 5′ UTR, possibly suggesting a regulatory function of gene expression given the proximity to promoter and other regulatory regions. This is interesting, given that ARHGAP18 belongs to the RHO-TPASE family; members of this family may control aspects of synapse function. The ARHGAP18 gene products such as RHO-GTPases are linked to RAS and epiderma growth factor receptor (EGFR)-mediated proliferation, migration, and differentiation of forebrain progenitors. The IGP involving this ARHGAP18 SNP-DLPFC relationship in schizophrenia is intriguing, as schizophrenia has been linked to abnormal prenatal neurogenesis, especially in the prefrontal cortex.

Discussion

A problem common to both neuroimaging and genome-wide scans is the high dimensionality of the data, with hundreds of thousands of measurements included in the analyses. Intuitively, combining these two fields should compound the problem; however, the approach we provide decreases the dimensionality.

We used differential brain imaging activation patterns as our starting point, based on the assumption that important pathophysiological differences are revealed by brain imaging. We then determined the impact of genetic variation on these brain activation patterns. A GLM was applied to the imaging phenotype and GWS results, following application of computational biology approaches to determine more of the genetic annotation for significant IGPs. The novel Imaging Genetics analyses are proof of concept of the provided approach that included massively parallel analyses of all 109,000 SNPs in conjunction with summary imaging results.

We have provided a demonstration of a new approach to identifying genes that are involved in brain function. The results above indicate the feasibility of these analyses on genome-wide scans. Although these results are intriguing, their role was as a training set on which to establish analysis and data reduction methods.

The following features can also be incorporated into the methods herein.

Brain imaging has been used to reveal the function of candidate genes, e.g. COMT. Our approach inverts the strategy that begins with a candidate gene and explores its effects on various phenotypes. Our statistical approach is built upon a general linear model that combines imaging phenotypes, disease diagnosis, and genetic data in a single model:

Imaging Phenotype=Genotype Effect+Diagnosis Effect+Genotype-Diagnosis Interaction Effect.

The value of this general method is that it includes the diagnosis by genotype interaction, as well as the ability to add additional terms for gene-gene interactions.

In the full method, we initially contrast brain imaging patterns between the patient population and normal healthy controls, to generate summary measures on differential activation patterns. A GLM parallel analyses of all SNPs is calculated with the brain activation measure as the dependent variable. The resultant IGPs are considered in a hierarchical procedure. Candidate genes determined a priori are first considered with a rigorous correction for the number of tests. Then the remaining SNPs (non-candidate) are considered using an appropriate corrections for a larger number of GLM tests. This procedure identifies top candidate genes and IGPs for further analysis.

Any method of correction based on statistical methods only brings with it an expected false negative rate. Additional genetic information will be expected to protect against false negatives, as well as removing false positives. Therefore, the SNPs that pass the rigorous correction above should be interrogated using a denser SNP chip; however, SNPs which failed the correction but showed a similar degree of significance should also be interrogated.

The identified genes from the above analyses are interrogated with a denser SNP chip to obtain additional information on genotyping in what can be considered a within-study confirmation. This censored analysis is repeated with the additional SNP data. The surviving results should be confirmed in an independent sample, which is essentially a between-study confirmation.

The first hierarchical analysis step in the independent confirmatory sample will be restricted to the positive results from the initial analysis in the original data set of candidate and non candidate IGPs that remain significant after further analysis with the denser chip data. It should be noted that the denser analysis can also contain VNTR and microsatellite or sequencing data.

The corrections for multiple testing at each stage is an ongoing point of research. We offered the most conservative Bonferroni in our example, although we acknowledge that the assumptions of independence have not been met and other corrections may be more appropriate. Other more recent methods to correct for the risk of falsely concluding for a positive association, i.e. increasing the risk of the frequency of False Positives, range from the Benjamini-Hochberg proposal (1995; 1997) adapted for genome analyses by Storey and Tibshirami (2003) with their FDR “correction” to the Nyholt (ref AJHG) and Meng (2003, AJHG) methods that consider the dependency across SNPs. Some methods however, like Nyholt's and Meng's, are well-suited for a “small” SNP set, e.g. as SNPs across a gene or in a chromosomal region, but are not easily generizable to whole genome association studies. Other methods provide to establish a sample-based significance threshold by a permutation approach (refs). Thus, at present we are still awaiting for a definitive approach that could appropriately correct for multiple testing, both considering the number of SNPs and their reciprocal dependency, without forgetting that any correction for type I errors should be traded-off with the risk of increasing the False Negative results.

In an initial attempt to decrease false positives, we introduced the criterion of “nearest neighbor” to constrain—and weight—the finding of significant SNPs: according to this principle, several of the SNPs within a gene should show evidence of an association with the phenotype. If only one SNM shows such an association, it is more likely to be a false positive.

A useful criterion, is the definition and classification of neighbor SNPs. One option is to require that the SNPs belong to the same haplotype block, but this conflicts with the independency of SNPs required for analysis. If they are not within the same haplo-block, they may be close enough to each other to be part of the same small chromosomal region, that may or may not overlap with any given (known) gene. In the latter case, SNPs fit the principle of independence, but their biological meaning may be ambiguous, especially in non-coding regions, where it is not clear what the SNPs are proxy for. The simplest way to adjust for “neighboring SNPs” is using a haplotyping approach, which is a well-known and accepted method (refs) despite some criticisms (e.g., terwilliger 2005). A haplotype-based “correction” will fulfill the criterion of independency of tests since each haplotype block is mostly independent from adjacent (nearby) blocks; thus correcting for the number of blocks rather than SNMs, even when considering single SNP testing, may be appropriate.

Of the 109K SNPs, 40-50% of genes are expressed in the brain (assuming 22-25K genes). A priori, any or all of these could correlate with brain activation. Empirically, we have determined that this is a relatively rare event in this dataset, with only 13 IGPs passing the criterion. Most of the analyzed SNPs were not related to brain activation in this task with this dataset. Further, investigation of gene annotation shows all the SNPs identified are expressed in the brain, an unexpected finding suggesting this is not random. This provides additional face validity to the finding, as there was a 60% chance of finding a SNP related to brain activity which is not expressed in the brain. We expected to find some clearly spurious results identifying genes that are not expressed in the brain.

The full method begins with an imaging phenotype which distinguishes subject groups. However, in this particular proof of concept example which focuses on the use of imaging phenotype to identify IGPs, there is no diagnostic term or interaction. We begin with summary statistics for the imaging results. In this particular example we picked an ROI based on known effects from the published literature.

The results of this proof of concept example are intriguing in several ways. Brain regions connected to left BA 46 also showed a significant influence of ARHGAP18 SNPs on brain activation measures, as shown in the FIGURE. These areas have several interesting features in common; all are neocortical regions that receive a dense dopamine innervation, all are highly interconnected, and participate in a dorsal cortical circuitry that is consistently implicated in the etiology of schizophrenia, especially the DLPFC. Interestingly, these areas are associated with dopamine function especially of the D1 receptors. Additionally, ARHGAP18 is precisely contained within 6q22-24, which has been shown to be linked to schizophrenia.

Results may still be false positives. Replication is desirable, either on a separate, independent sample or through more thorough investigation of the mechanism by which the identified SNPs may influence the illness. The latter can include gene sequencing and animal studies, and other functional genetic studies at the molecular and cellular levels.

The genome-wide scan does not pick up all possible SNPs nor types of variation, so gene-sequencing around identified SNPs is warranted. Any findings here may not be unique to schizophrenia, given the lack of a control group. The point of these results, however, is the application of the method rather than a definitive diagnostically-related genetic influence.

This approach is a screening method that makes GWS data usable and exploratory in preparation for future studies, e.g. molecular studies, expression and transgenic studies, and all other functional genomic approaches. It allows for completely novel SNPs to be identified as playing a role in the disease phenotype.

ARHGAP 18

The ARHGAP18 gene products are Rho GTPases. They belong to the Ras superfamily which is composed of over fifty members divided into six families, including Ras, Sar, Rho, Ran, Rab, and Arf (Takai et al. 2001). They participate in an array of physiological processes, such as cell migration, intercellular adhesion, cytokinesis, proliferation, differentiation and apoptosis (Symons et al 1996). The proteins exist in two interconvertible forms: the GDP-bound inactive form and the GTP-bound active form. The Rho proteins act as molecular switches which might turn on or off a regulated group of signaling pathways. The switch between the active state, bound to GTP, and inactive state, bound to GDP, is controlled by several types of regulatory factors. Active GTPases interact with downstream targets to effect their cellular functions, whereas GTP-hydrolysis and release of phosphate inactivate the GTPases. Rho GTPases are important regulators of the actin cytoskeleton and consequently influence the shape and movement of the cells. GTPases of the Rho family are strong regulators of signaling pathways that link growth factors and/or their receptors to adhesions and associated structures (Kozma et ai. 1995). GTPases in the Rho family also regulate cadherin-mediated intercellular adhesion (Braga et al. 1999), one of which is p120-catenin which binds cadherin and promotes its clustering with RhoA, which enhances adhesion. By regulating RhoA activation, p120ctn modulates cadherin functions, including neurite extension and intercellular junction formation (Noren et al. 2000). One signaling pathway mediated by Ras is initiated by the epidermal growth factor (EGF) receptor (EGFR) leading to cell proliferation. EGFR signaling can induce mitosis, proliferation, cell motility, differentiation, and protein secretion (Wells 1999). EGFR is localized on subventricular neural progenitors in the fetal and adult lateral ventricles, and these progenitors give rise to forebrain neurons in development and after injury in the adult (see Fallon et al 2000). Thus, the ARHGAP18 gene products (Rho GTPases) are linked to Ras and thus to EGFR-mediated proliferation, migration and differentiation of forebrain progenitors. Therefore, our finding of a ARHGAP18 SNPs-DLPFC IGP in schizophrenia is interesting because schizophrenia has been linked to altered prenatal neurogenesis of cortical neurons, including those in dorsal prefrontal cortex.

The invention optionally includes manipulating this gene and its gene products to both alter the onset and course of schizophrenia, and also create animal models of schizophrenia by, for example treating prenatal and perinatal animals and also the gravid mothers with ARHGAP18 antisense. The expression of this gene and/or its polymorphisms or other expression variations can be used to diagnosis high risk individuals, prodromal and ill subjects.

Although the above discussion has presented the present invention according to specific methods, systems, compositions, kits and apparatus, the present invention has a broader range of applicability. Further, while the foregoing invention has been described in some detail for purposes of clarity and understanding, it will be clear to one skilled in the art from a reading of this disclosure that various changes in form and detail can be made without departing from the true scope of the invention. For example, all the methods, techniques, systems, devices, kits, apparatus, etc., described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

| # | SNP | Chromosome | Coordinate | CytogeneticBand | GeneticMapPosition | GeneSymbol | RefSeqGene | RefSeqLocation |
|---|---|---|---|---|---|---|---|---|
| 1987 | rs2244008 | chr6 | 129854746 | 6q22.33 | 128.939 | LAMA2 | NM_000426 | coding |
| 5713 | rs9321170 | chr6 | 129864530 | 6q22.33 | 128.9514 | LAMA2 | NM_000426 | intron |
| 2200 | rs2297740 | chr6 | 129877507 | 6q22.33 | 128.9679 | LAMA2 | NM_000426 | intron |
| 570 | rs12197456 | chr6 | 129946942 | 6q22.33 | 129.0561 | ARHGAP18 | NM_033515 | coding |
| 6453 | rs9492347 | chr6 | 129948346 | 6q22.33 | 129.0579 | ARHGAP18 | NM_033515 | intron |
| 643 | rs12530181 | chr6 | 129966696 | 6q22.33 | 129.0812 | ARHGAP18 | NM_033515 | intron |
| 6111 | rs9388717 | chr6 | 129978524 | 6q22.33 | 129.0962 | ARHGAP18 | NM_033515 | intron |
| 5997 | rs9375644 | chr6 | 129993694 | 6q22.33 | 129.1194 | ARHGAP18 | NM_033515 | intron |
| 206 | rs10499163 | chr6 | 130004326 | 6q22.33 | 129.1427 | ARHGAP18 | NM_013515 | intron |
| 5965 | rs9372944 | chr6 | 130007047 | 6q22.33 | 129.1486 | ARHGAP18 | NM_033515 | intron |
| 1603 | rs2051632 | chr6 | 130050625 | 6q22.33 | 129.244 | ARHGAP18 | NM_033515 | intron |
| 3052 | rs3752536 | chr6 | 130072908 | 6q22.33 | 129.2928 | ARHGAP18 | NM_033515 | coding |
| 3913 | rs4897338 | chr6 | 130128515 | 6q22.33 | 129.4145 | ARHGAP18 | NM_033515 | flanking_5UTR |
| 3914 | rs4897344 | chr6 | 130158851 | 6q22.33 | 129.4809 | ARHGAP18 | NM_033515 | flanking_5UTR |
| 5411 | rs7776426 | chr6 | 130194213 | 6q22.33 | 129.5583 | ARHGAP18 | NM_033515 | flanking_5UTR |
| 963 | rs1480513 | chr6 | 130207677 | 6q22.33 | 129.5878 | ARHGAP18 | NM_033515 | flanking_5UTR |
| 6084 | rs9385523 | chr6 | 130209861 | 6q22.33 | 129.5925 | ARHGAP18 | NM_Q33515 | flanking_5UTR |
| 6190 | rs9398929 | chr6 | 130281084 | 6q22.33 | 129.7484 | L3MBTL3 | NM_001007102 | flanking_5UTR |
| 5211 | rs7754426 | chr6 | 130372958 | 6q22.33 | 129.9295 | L3MBTL3 | NM_001007102 | flanking_5UTR |
| 3363 | rs3890746 | chr6 | 130412748 | 6q23.1 | 129.9576 | L3MBTL3 | NM_032438 | flanking_3UTR |

| # | SNP | RefSeqLocationRelativeToGene | EnsemblGene | EnsemblLocation |
|---|---|---|---|---|
| 1987 | rs2244008 | [7/169] | ENST00000354729 | coding |
| 5713 | rs9321170 | −967 | ENST00000354729 | flanking_5UTR |
| 2200 | rs2297740 | −74 | ENST00000355250 | flanking_3UTR |
| 570 | rs12197456 | [116/8] | ENST00000275189 | coding |
| 6453 | rs9492347 | −1396 | ENST00000275189 | intron |
| 643 | rs12530181 | −2019 | ENST00000275189 | intron |
| 611 | rs9388717 | −526 | ENST00000275189 | intron |
| 5997 | rs9375644 | −1334 | ENST00000275189 | intron |
| 206 | rs10499163 | −328 | ENST00000275189 | intron |
| 5965 | rs9372944 | −2191 | ENST00000275189 | intron |
| 1603 | rs2051632 | −22237 | ENST00000275189 | intron |
| 3052 | rs3752536 | [46/66] | ENST00000275189 | coding |
| 3913 | rs4897338 | −55452 | ENST00000275189 | flanking_5UTR |
| 3914 | rs4897344 | −85788 | ENST00000345007 | flanking_3UTR |
| 5411 | rs7776426 | −121150 | ENST00000345007 | coding |
| 963 | rs1480513 | −134614 | ENST00000345007 | intron |
| 6084 | rs9385523 | −136798 | ENST00000345007 | intron |
| 6190 | rs9398929 | −100343 | ENST00000345007 | flanking_5UTR |
| 5211 | rs7754426 | −8469 | ENST00000354350 | flanking_5UTR |
| 3363 | rs3890746 | −80 | ENST00000354350 | intron |

| # | SNP | EnsemblLocationRelativeToGene | SWISS-PROTGene | SWISS-PROTLocation | SWISS-PROTLocationRelativeToGene |
|---|---|---|---|---|---|
| 1987 | rs2244008 | [7/169] | LMA2_HUMAN | coding | [7/169] |
| 5713 | rs9321170 | −967 | LMA2_HUMAN | intron | −967 |
| 2200 | rs2297740 | −74 | LMA2_HUMAN | intron | −74 |
| 570 | rs12197456 | [116/8] | Q96S64 | coding | [116/8] |
| 6453 | rs9492347 | −1396 | Q96S64 | intron | −1396 |
| 643 | rs12530181 | −2019 | Q6PJD7 | flanking_3UTR | −2019 |
| 6111 | rs9388717 | −526 | Q96S64 | intron | −526 |
| 5997 | rs9375644 | −1334 | Q96S64 | intron | −1334 |
| 206 | rs10499163 | −328 | Q8N392 | flanking_3UTR | −328 |
| 5965 | rs9372944 | −2191 | Q8N392 | flanking_5UTR | −2191 |
| 1603 | rs2051632 | −22237 | Q6P679 | flanking_3UTR | −22237 |
| 3052 | rs3752536 | [46/93] | Q6P679 | coding | [46/64] |
| 3913 | rs4897338 | −55452 | Q8N392 | flanking_5UTR | −55514 |
| 3914 | rs4897344 | −35306 | Q8N392 | flanking_5UTR | −85850 |
| 5411 | rs7776426 | [56/11] | Q8N392 | flanking_5UTR | −121212 |
| 963 | rs1480513 | −928 | Q8N392 | flanking_5UTR | −134676 |
| 6084 | rs9385523 | −1171 | Q8N392 | flanking_5UTR | −136860 |
| 6190 | rs9398929 | −57058 | Q6P9B5 | flanking_5UTR | −100343 |
| 5211 | rs7754426 | −8469 | Q6P9B5 | flanking_5UTR | −8469 |
| 3363 | rs3890746 | −80 | Q96JM7 | flanking_3UTR | −80 |

| # | SNP | Coding_Status | AAChange (Gene) | PhastConsElementsScore | MouseIdentity |
|---|---|---|---|---|---|
| 1987 | rs2244008 | NONSYN | T2636A(NP_000417) | 20 | 0.83 |
| 5713 | rs9321170 | | | | 0.7 |
| 2200 | rs2297740 | | | | 0.79 |
| 570 | rs12197456 | SYNON | | 176 | 0.95 |
| 6453 | rs9492347 | | | | |
| 643 | rs12530181 | | | | |
| 6111 | rs9388717 | | | 38 | 0.87 |
| 5997 | rs9375644 | | | | |
| 206 | rs10499163 | | | | |
| 5965 | rs9372944 | | | | |
| 1603 | rs2051632 | | | | |
| 3052 | rs3752536 | NONSYN | T23A(NP_277050) | 75 | 0.84 |
| 3913 | rs4897338 | | | | |
| 3914 | rs4897344 | | | | |
| 5411 | rs7776426 | NONSYN | F111V(XP_173166) | 32 | 0.88 |
| 963 | rs1480513 | | | 28 | |
| 6084 | rs9385523 | | | 22 | 0.82 |
| 6190 | rs9398929 | | | 86 | 0.86 |
| 5211 | rs7754426 | | | | |
| 3363 | rs3890746 | | | | 0.78 |

| Chromosome | Gene | Name |
|---|---|---|
| chr1 | LOC148823 | |
| | [C1orf150] | unknown |
| chr2 | PPP1CB | protein phosphatase 1, catalytic subunit, beta isoform |
| chr2 | SPDY1 | speedy homolog A (Drosophila) |
| chr2 | LRP1B | low density lipoprotein-related protein 1B (deleted in tumors) |
| chr2 | PLA2R1 | phospholipase A2 receptor 1, 180 kDa |
| chr2 | KIAA1604 | |
| chr2 | COL4A3 | collagen, type IV, alpha 3 (Goodpasture antigen) |
| chr2 | MGC42174 | |
| chr3 | IGDF4D | Immunoglobulin superfamily, member 4D |
| chr3 | MGC12197 | arginine/serine-rich coiled-coil 1 |
| | (new = RSRC1) | |
| chr4 | PITX2 | paired-like homeodomain transcription factor 2 |
| chr4 | NPY5R | neuropeptide Y receptor Y5 |
| chr5 | ZNF608 | zinc finqer protein 608 |
| chr5 | SFXN1 | sideroflexin 1 |
| chr6 | ARHGAP18 | Rho GTPase activating protein 18 |
| chr8 | ARHGEF10 | Rho guanine nucleotide exchange factor (GEF) 10 |
| chr8 | ZFPM2 | zinc finger protein, multltype 2 |
| chr9 | SLC24A2 | solute carrier family 24 (sodium/potassium/calcium exchanger), member 2 |
| chr9 | ZNF297B | zinc firmer and BTB domain containing 43 |
| chr10 | MKI67 | antigen identified by monoclonal antibody KI-67 |
| chr11 | FLJ22531 | hypothetical protein FLJ22531 |
| chr11 | PC | pyruvate carboxylase |
| chr11 | ZNF195 | zinc finger protein 195 |
| chr12 | LOC387882 | hypothetical protein LOC387882 |
| chr13 | FLJ40296 | FLJ40296 protein |
| chr16 | SPINL | spinster (???) |
| | (new = SPIN1) | |
| chr16 | CHIP | c-Maf-inducing protein |
| chr16 | DKFZP434B044 | cysteine-rich secretory protein LCCL domain containing 2 |
| | (new = CRISPLD2) | |

| Chromosome | Gene | GeneCards | | |
|---|---|---|---|---|
| chr1 | LOC148823 | | | |
| | [C1orf150] | http://www.genecards.org/cgi-bin/carddisp.pl?gene=C1orf150 | | |
| chr2 | PPP1CB | http://www.genecards.org/cgi-bin/carddisp.pl?gene=PPP1CB | | |
| chr2 | SPDY1 | SPDY1 | | |
| chr2 | LRP1B | http://www.genecards.org/cgi-bin/carddisp.pl?gene=LRP1B | | |
| chr2 | PLA2R1 | http://www.genecards.org/cgi-bin/carddisp.pl?gene=PLA2R1 | | |
| chr2 | KIAA1604 | KIAA1604 protein from NCBI | | |
| chr2 | COL4A3 | http://www.genecards.org/cgi-bin/carddisp.pl?gene=COL4A3 | | |
| chr2 | MGC42174 | hypothetical protein MGC42174 - from NCBI | | |
| chr3 | IGDF4D | http://www.genecards.org/cgi-bin/carddisp.pl?gene=IGSF4D | | |
| chr3 | MGC12197 | http://www.genecards.org/cgi-bin/carddisp.pl?gene=RSRC1 | | |
| | (new = RSRC1) | | | |
| chr4 | PITX2 | http://www.genecards.org/cgi-bin/carddisp.pl?gene=PITX2 | | |
| chr4 | NPY5R | http://www.genecards.org/cgi-bin/carddisp.pl?gene=NPY5R | | |
| chr5 | ZNF608 | http://www.genecards.org/cgi-bin/carddisp.pl?gene=ZNF608 | | |
| chr5 | SFXN1 | http://www.genecards.org/cgi-bin/carddisp.pl?gene=SFXN1 | | |
| chr6 | ARHGAP18 | http://www.genecards.org/cgi-bin/carddisp.pl?gene=ARHGAP18 | | |
| chr8 | ARHGEF10 | http://www.genecards.org/cgi-bin/carddisp.pl?gene=ARHGEF10 | | |
| chr8 | ZFPM2 | http://www.genecards.org/cgi-bin/carddisp.pl?gene=ZFPM2 | | |
| chr9 | SLC24A2 | http://www.genecards.org/cgi-bin/carddisp.pl?gene=SLC24A2 | | |
| chr9 | ZNF297B | http://www.genecards.org/cgi-bin/carddisp.pl?gene=ZNF297B&search=ZNF297B | | |
| chr10 | MKI67 | http://www.genecards.org/cgi-bin/carddisp.pl?gene=MKI67 | | |
| chr11 | FLJ22531 | http://www.ncbi.nim.nih.gov/entrez/query.fcgi?db=gene&cmd=Retrieve&dopt=full_report&list_uids=79703 - | | |
| | | pageTop | | |
| chr11 | PC | http://www.genecards.org/cgi-bin/carddisp.pl?gene=PC | | |
| chr11 | ZNF195 | http://www.genecards.org/cgi-bin/carddisp.pl?gene=ZNF195 | | |
| chr12 | LOC387882 | http://www.ncbi.nim.nih.gov/entrez/query.fcgi?db=gene&cmd=Retrieve&dopt=full_report&list_uids=387882 | | |
| chr13 | FLJ40296 | http://www.ncbi.nim.nih.gov/entrez/query.fcgi?db=gene&cmd=Retrieve&dopt=full_report&list_uids=122183 | | |
| chr16 | SPINL | http://www.ncbi.nim.nih.gov/entrez/query.fcgi?db=gene&cmd=Retrieve&dopt=full_report&list_uids=83985 | | |
| | (new = SPIN1) | | | |
| chr16 | CHIP | http://www.ncbi.nim.nih.gov/entrez/query.fcgi?db=gene&cmd=Retrieve&dopt=full_report&list_uids=80790 | | |
| chr16 | DKFZP434B044 | http://www.genecards.org/cgi-bin/carddisp.pl?gene=CRISPLD2 | | |
| | (new = CRISPLD2) | | | |
| Chromosome | Gene | Function | Pathway | Additional info |
| chr1 | LOC148823 | unknown | | |
| | [C1orf150] | | | |
| chr2 | PPP1CB | ++++++ Protein phosphatase (PP1) is | 4 KEGG Pathways & 5 | |
| | | essential for cell division, it participates in the | Invitrogen Pathways (see | |
| | | regulation of glycogen metabolism, muscle | GeneCard) | |
| | | contractility and protein synthesis. Involved in | | |
| | | regulation of ionic conductances and long-term | | |
| | | synaptic plasticity | | |
| chr2 | SPDY1 | unknown | | |
| chr2 | LRP1B | Potential cell surface proteins that bind and | | |
| | | internalize ligands in the process of | | |
| | | receptor-mediated endocytosis | | |
| chr2 | PLA2R1 | | | |
| chr2 | KIAA1604 | | | |
| chr2 | COL4A3 | Type IV collagen is the major structural | | |
| | | component of glomerular basement membranes | | |
| | | (GBM), forming a ‘chicken-wire’ meshwork | | |
| | | together with laminins, proteoglycans and | | |
| | | entactin/nidogen | | |
| chr2 | MGC42174 | http://www.ncbi.nim.nih.gov/entrez/batchseq.cgi | | |
| | | ?db=popset&view=ps&val=66887648 | | |
| chr3 | IGDF4D | Immunoglobutin - Cell Adhesion | | |
| chr3 | MGC12197 | Physically interacts with GDF9 −> Growth | | |
| | (new = RSRC1) | differentiation facotr 9 precursir | | |
| chr4 | PITX2 | May play an important role in development | TGF-beta signalling pathway - | GeneDecks results for |
| | | and maintenance of anterior structures. | Homo sapiens (human) | genes in the same |
| | | Isoform PTX2C is involved in left-right | | KEGG pathways as |
| | | asymmetry the developing embryo | | PITX2 (# = 85) |
| chr4 | NPY5R | Receptor for neuropeptide Y and peptide YY. The | Neuroactive ligand-receptor | GeneDecks results for |
| | | activity of this receptor is mediated byG proteins | interaction - Homo sapiens | genes in the same |
| | | that inhibit adenylate cyclase activity. Seems to | (human) | KEGG pathways as |
| | | be associated with food intake. Could be involved | | NPY5R (# = 224) |
| | | in feeding disorders | | |
| chr5 | ZNF608 | | | |
| chr5 | SFXN1 | Might be involved in the transport of a component | | Interact with IKBKG |
| | | required for iron utilization into or | | |
| | | out of the mitochondria | | |
| chr6 | ARHGAP18 | | | |
| chr8 | ARHGEF10 | May play a role in developmental myelination of | | |
| | | peripheral nerves | | |
| chr8 | ZFPM2 | Transcription regulator that plays a central role in | | |
| | | heart morphogenesis and development | | |
| | | of coronary vessels from epicardium, by | | |
| | | regulating genes that are essential during | | |
| | | cardiogenesis. | | |
| | | Essential cofactor that acts via the formation of a | | |
| | | heterodimer with transcription factors of the | | |
| | | GATA family GATA4, GATA5 and GATA6. Such | | |
| | | heterodimer can both activate or repress | | |
| | | transcriptional | | |
| | | activity, depending on the cell and promoter | | |
| | | context. Also required in gonadal differentiation, | | |
| | | possibly be regulating expression of SRY | | |
| chr9 | SLC24A2 | Critical component of the visual transduction | | |
| | | cascade, controlling the calcium | | |
| | | concentration of outer segments during light and | | |
| | | darkness. Light causes a rapid lowering of | | |
| | | cytosolic free calcium in the outer segment of | | |
| | | both retinal rod and cone photoreceptors and the | | |
| | | light-Induced lowering of calcium is caused by | | |
| | | extrusion via this protein which plays a key role | | |
| | | in the process of light adaptation. Transports 1 | | |
| | | Ca(2+) and 1 K(+) in exchange for 4 Na(+) | | |
| chr9 | ZNF297B | | | |
| chr10 | MKI67 | Asymmetrical cell division? | | interacts with protein |
| | | Thought to be required for maintaining cell | | of unknown function |
| | | proliferation | | |
| chr11 | FLJ22531 | unknown | | |
| chr11 | PC | Pyruvate carboxylase catalyzes a 2-step reaction, | 3 pathways (seeGeneCard): | |
| | | Involving the ATP-dependent | 1 = CITRATE CYCLE (TCA | |
| | | carboxylation of the covalently attached biotin in | CYCLE) - 2 = Alanine and | |
| | | the first step and the transfer of the carboxyl | aspartate metab - 3 = | |
| | | group to pyruvate in the second. Catalyzes in a | Pyruvate metab | |
| | | tissue specific manner, the initial reactions of | | |
| | | glucose (liver, kidney) and lipid (adipose tissue, | | |
| | | liver, brain) synthesis from pyruvate | | |
| chr11 | ZNF195 | May be involved in transcriptional regulation | | |
| chr12 | LOC387882 | uhknown | | |
| chr13 | FLJ40296 | unknown | | |
| chr16 | SPINL | Spinster protein interferes with, programmed cell | | |
| | (new = SPIN1) | death in Drosdphila melanogaster and has | | |
| | | orthologs in nematode, mouse, and human. | | |
| chr16 | CHIP | results suggest that Tc-mip plays a critical role in | Filamin-A interacts with c- | |
| | | Th2 signaling pathway and represents the first | mip/Tc-mip in a new T-cell | |
| | | proximal signaling protein which links TCR- | signaling pathway. | |
| | | mediated signal to the activation of c-maf Th2 | | |
| | | specific factor | | |
| chr16 | DKFZP434B044 | unknown | | |
| | (new = CRISPLD2) | | | |

1-21. (canceled)

22. A method of correlating a brain image phenotype to a genotype, the method comprising:
detecting variance in a brain image phenotype in at least one population;
accessing genotype information for the population; and,
correlating the variance to the genotype information, thereby correlating the brain image phenotype and the genotype.

23. The method of claim 22, wherein the population comprises a group of cogitatively and psychiatrically healthy individuals and a group of patients that suffer from a neuropsychiatric disorder, and the variance is a difference in brain image phenotype between the normal individuals and the patients.

24. The method of claim 23, wherein the group of patients that suffer from a neuropsychiatric disorder comprise patients that are schizophrenic or that suffer from a bipolar disorder.

25. The method of claim 23, wherein the brain image comprises an fMRI brain scan of the patient.

26. The method of claim 22, wherein the fMRI comprises a functional MRI test of the normal and abnormal patients, the functional MRI test comprising a working memory test.

27. The method of claim 22, wherein the variance in the brain image phenotype comprises a variance in differential brain activation between members of the population.

28. The method of claim 22, wherein detecting variance in a brain image phenotype comprises assigning a summary statistic for an image for at least one region of the brain for at least one member of the population.

29. The method of claim 28, wherein assigning the summary statistic comprises: measuring a first brain image of a brain region under a first functional condition;
measuring a second brain image of the brain region under a second functional condition;
determining a difference between the first and second brain image; and,
assigning the summary statistic to reflect the difference.

30. The method of claim 28, wherein the first brain image and the second brain image are extracted from a corresponding first and second brain scan using a Talairach or MNI atlas.

31. The method of claim 28, wherein the summary statistic reflects a difference between an observed brain image for a brain engaged in a high memory task and an observed brain image for a brain engaged in a low memory task for the at least one region.

32. The method of claim 28, wherein the at least one region is selected from the group consisting of: the left hemisphere Broadman Area 46, DLPFC BA-9, DPFC, BA 6 the Premotor Cortex, the Dorsal Premotor Cortex, BA 7 (Superior Parietal Lobule), BA 8 Frontal Eye Field/Premotor Cortex, posterior dorsal prefrontal cortex, BA24 (Left Anterior Cingulate), the Left Whole Thalamus, Caudate, Amygdala, and the Right Cerebellum.

33. The method of claim 22, wherein the genotype information comprises a dataset derived from hybridization of a sample to an array of polymorphisms.

34. The method of claim 22, wherein the genotype information comprises SNP data sets for at least about 100,000 representative SNPs for a plurality of members of the population.

35. The method of claim 22, wherein the variance is correlated using a general linear model.

36. The method of claim 35, wherein the general linear model assumes that imaging phenotype=overall mean+genotype effect+diagnosis effect+genotype-diagnosis interaction effect.

37. The method of claim 22, wherein the variance is correlated by performing linear regression to compare image phenotype information across the population to SNP genotype information across the population, wherein the comparison comprises testing for an equality of means across the genotype information, assuming a codominant genetic model that tests for additive effects, dominant effects and effects equal to zero.

38. The method of claim 22, wherein the variance is correlated to genetically linked polymorphisms using a haplotype correction criterion.

39. The method of claim 22, wherein the variance is correlated to a plurality of genetically linked polymorphisms using a within-study confirmation analysis.

40. The method of claim 22, wherein the variance is a first variance in differential activation in a first region of the brain, and the method comprises detecting an additional variance in differential activation in an anatomically or functionally connected region of the brain, and wherein the first variance and the additional variance correlate similarly to the genotype information.

41. The method of claim 22, further comprising replicating the correlation in an independent sample or population.

42-50. (canceled)
